(19) United States
(12) Patent Application Publication
(10) Pub. No.: US 2021/0094744 A1
(43) Pub. Date: Apr. 1, 2021

(54) PRIMARY PACKAGE CONTAINING PERSONAL CARE ARTICLE

(71) Applicant: The Procter & Gamble Company, Cincinnati, OH (US)

(72) Inventors: William Mercer Benson, Harrison, OH (US); Douglas Charles Cook, South Lebanon, OH (US); Scott David Hochberg, Cincinnati, OH (US); Pamela Ann Keune, West Chester, OH (US)

(21) Appl. No.: 16/589,504

(22) Filed: Oct. 1, 2019

(51) Int. Cl. B65D 77/20; B32B 27/36; B65D 75/58; B32B 15/20; A61K 8/02

(52) U.S. Cl. CPC B65D 77/2032; B32B 27/36; B65D 75/5838; A61K 2800/87; A61K 8/0216; B65D 2577/205; B32B 15/20

(57) ABSTRACT

A personal care product comprising a primary package with a thermoform portion and a lidding film. The thermoform portion includes a peripheral flange and the lidding film is peelably sealed to the peripheral flange. The primary package can comprise a moisture vapor transmission rate of less than 0.25 g/m²/day. The thermoform portion is adapted to hold a solid personal care article and the primary package can protect the personal care article from compression.

### FIELD OF THE INVENTION

The present invention is generally directed to primary packaging, and more particular primary packaging adapted to contain a solid personal care article.

### BACKGROUND OF THE INVENTION

Many personal care and other consumer products, including shampoos, conditioners, and body washes, in the market today are sold in liquid form. While widely used, liquid personal care products often have tradeoffs. For example, liquid personal care products are typically sold in plastic bottles that add significant cost as well as packaging waste. Also, these products are generally formulated with a substantial amount of water (e.g. −80% or more), preservatives, and stabilizers, that add significant bulk and translates to inefficient, costly shipping and storage. Furthermore, liquid personal care products can also be difficult to use in terms of controlling dosage and the delivery of the product.

In order to overcome some of these drawbacks, it can be desirable to formulate personal care products as solid structures that can include dissolvable films, compressed powders in a solid, fibrous structures, porous foams, soluble deformable solids, powders, bars or prills. However, many of these executions are not ideal for consumers. For example, some products including many bars or prills, do not hydrate and dissolve fast enough when exposed to water to satisfy the consumer's desire to quickly apply a homogeneous liquid product to the hair, scalp, and/or body, without undue effort to dissolve the product.

Solid articles containing fibrous structures or porous foams can be lightweight, substantially free of water, and can provide faster dissolution than other solid forms. However, shipping, handling, and storage can damage these articles. First, many articles containing fibrous structures or porous films can be hygroscopic and therefore easily absorb humidity from the environment. If the product absorbs too much water it can be a gummy mess or alternatively, become difficult to dissolve with water. This makes storage problematic, since personal care products, including shampoos, conditioners, and body washes, are frequently stored in the shower or bathroom, they are regularly subjected to water and humidity. Furthermore, articles containing fibrous structures and/or porous foams are compressible, making shipping, handling, and storage problematic.

Therefore, there is a need for a primary package for an article formed from a fibrous structure or porous foam that protects the article from moisture, compression, and where the thermoform portion is recyclable.

### SUMMARY OF THE INVENTION

A personal care product comprising: (a) primary package comprising: (i) a thermoform portion comprising a peripheral flange and a thermoform compartment; wherein the thermoform portion is comprised of a thermoform material selected from the group consisting of polypropylene, polyethylene terephthalate, polyethylene terephthalate glycol, poly-chloro-trifluoroethylene/polyethylene terephthalate glycol, and combinations thereof; (ii) a lidding film peelably sealed to the peripheral flange; wherein the primary package comprises a moisture vapor transmission rate of less than 0.25 g/m²/day; (b) a solid fibrous article comprising filaments comprising: (i) from about 1 wt % to about 50 wt % of a polymeric structurant having a weight average molecular weight of from about 10,000 to about 6,000,000 g/mol; (ii) a surfactant selected from the group consisting of anionic surfactants, cationic surfactants, zwitterionic surfactants, and combinations thereof; wherein the fibrous article fits within the thermoform compartment; wherein the fibrous article compresses less than 30% under a compressive force of at least 15 psi; (c) a headspace disposed between the lidding layer and a top surface of the solid fibrous article wherein the headspace comprises a height of from about 5% to about 30%, of the height of the thermoform.

A personal care product comprising: (a) primary package comprising: (i) a thermoform portion comprising a peripheral flange and a thermoform compartment; wherein the thermoform portion is comprised of a thermoform material selected from the group consisting of polypropylene, polyethylene terephthalate, polyethylene terephthalate glycol, poly-chloro-trifluoroethylene/polyethylene terephthalate glycol, and combinations thereof; (ii) a lidding film peelably sealed to the peripheral flange with a heat seal; wherein the lidding film comprises a multilayer laminate wherein at least one layer is selected from the group consisting of aluminum, polyethylene terephthalate, and combinations thereof; wherein the primary package comprises a moisture vapor transmission rate of less than 0.20 g/m²/day; (b) a solid article comprising a surfactant; wherein the fibrous article fits within the thermoform compartment; (c) a headspace disposed between the lidding layer and a top surface of the solid article wherein the headspace comprises a height of from about 0.05 mm to about 1.5 mm; wherein the primary package protects the solid article from compressing more than 30% under a compressive force of 50 N.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

While the specification concludes with claims particularly pointing out and distinctly claiming the subject matter of the present invention, it is believed that the invention can be more readily understood from the following description taken in connection with the accompanying drawings, in which:

FIG. 1 is a first perspective schematic view of a primary package;

FIG. 2 is a second perspective schematic view of the primary package of FIG. 1;

FIG. 3A is an enlarged view of a pull tab where the lidding film is in plane with the flange;

FIG. 3B is an enlarged view of a pull tab where the lidding film is out of plane with the flange;

FIG. 3C is an enlarged view of a pull tab where the flange includes bumps;

FIG. 4 is a cross-sectional view of the primary package of FIGS. 1 and 2 taken along the plane of the line A-A;

FIG. 5 is an example of a solid personal care article;

FIG. 6 is a schematic representation of an example of a fibrous element according to the present invention;

FIG. 7 is a schematic representation of an example of a process for making fibrous elements of the present invention;

FIG. 8 is a schematic representation of an example of a die with a magnified view used in the process of FIG. 7;

FIG. 9A is a computerized tomography (CT) scan of a fibrous article of Example A, stored in a weigh boat;

FIG. 9B is a computerized tomography (CT) scan of a fibrous article of Example A, packaged and stored in a sachet;

FIG. 10A is a 3D computerized tomography (CT) scans of a fibrous article of Example A, stored in a weigh boat;

FIG. 10B is a 3D computerized tomography (CT) scans of a fibrous article of Example A, packaged and stored in a sachet;

FIG. 11 is a chart showing the % relative humidity vs. time for five different thermoforms; and

FIG. 12 is a chart showing the % relative humidity inside the thermoform compartment vs. time.

### DETAILED DESCRIPTION OF THE INVENTION

While the specification concludes with claims particularly pointing out and distinctly claiming the invention, it is believed that the present disclosure will be better understood from the following description.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise noted, all component or composition levels are in reference to the active level of that component or composition, and are exclusive of impurities, for example, residual solvents or by-products, which may be present in commercially available sources.

It can be desirable to form personal care articles, such as shampoo, conditioners, and body washes, in a solid form. A solid form can be advantageous for several reasons. First, solid products can be substantially free of water, preservatives, and stabilizers, making these products more efficient and cost effective to ship and store. Additionally, solid products, especially unit dose solid products, are easy to dose and can practically eliminate wasted product inside the primary package. Furthermore, consumers are looking for ways to reduce waste and plastic bottles add significant cost as well as packaging waste and consumers may be interested in plastic packaging that is recyclable.

For solid forms to appeal to consumers, they need to disintegrate and form a homogeneous solution soon after exposure to water. It was found that solid articles made primarily from fibrous structures and/or porous foams can provide consumer acceptable dissolution rates. A consumer acceptable dissolution rate can mean that the dissolvable solid structure can have a hand dissolution value (as determined by the Hand Dissolution Test Method, described hereafter) of less than about 20 strokes. Fibrous structures, as described in U.S. Pub. Nos. 2012/052036, 2018/0333339, and 2019/0282461, U.S. application Ser. No. 15/981,096, U.S. Pat. No. 9,545,364 and porous foams, as described in U.S. Pat. Nos. 8,349,786, 8,461,091, and 8,349,787 and US Pub. No. 2012/0270029, incorporated by reference, can be particularly attractive to consumers.

Solid articles containing fibrous structures or porous foams can be hygroscopic and therefore can easily absorb humidity from the environment. Since these articles are frequently stored in the bathroom or shower, which has a high relative humidity, as compared to other climate-controlled areas of a consumer's home, it is important that the primary package protects the article from the humidity. For example, a shower can have an average relative humidity of 68% at 68° F.

The primary package can have an MVTR (moisture vapor transmission rate) of less than 0.5 g/m²/day, alternatively less than 0.3 g/m²/day, alternatively less than 0.25 g/m²/day, alternatively less than 0.20 g/m²/day, alternatively less than 0.15 g/m²/day. The primary package can have an MVTR of from about 0.05 g/m²/day to about 0.7 g/m²/day, alternatively from about 0.07 g/m²/day to about 0.6 g/m²/day, alternatively from about 0.1 g/m²/day to about 0.5 g/m²/day, alternatively from about 0.1 g/m²/day to about 0.4 g/m²/day, alternatively from about 0.1 g/m²/day to about 0.3 g/m²/day, alternatively from about 0.1 g/m²/day to about 0.25 g/m²/day, alternatively from about 0.1 g/m²/day to about 0.2 g/m²/day, alternatively from about 0.1 g/m²/day to about 0.15 g/m²/day. MVTR can be determined by ASTM E398-13.

Furthermore, articles containing fibrous structures and/or porous foams are compressible. These articles are shipped, transported to the store/storage facility, and stored by the consumer. Since these articles are unit dose products, it is common for consumers to carry these products in their purse, gym bag, or suitcase, subjecting the article to further damage. The primary package can be subjected to about 18 to about 25 psi inside a purse or gym bag. It was found that if the article is compressed too much, it will not return to its original structure, which can significantly impact the dissolution rate. The dissolvable fibrous article can comprise a hand dissolution of less than or equal to 20 strokes, alternatively less than or equal to 15 strokes, alternatively less than or equal to 12 strokes, alternatively less than or equal to 10 stokes, and alternatively less than or equal to 8 strokes, after storage in the primary package for one week, two weeks, and/or six weeks, at 104° F. and 75% relative humidity, according to the Hand Dissolution Test Method.

It was found that if the pad is compressed too much during handling and storage, it will not have consumer acceptable dissolution (e.g. greater than 20 strokes), according to the Hand Dissolution Test Method, described herein.

The primary package can protect the solid article, so the article is compressed less than 2 mm (0.079 in.) during shipping, handling, and storage, alternatively less than 1.5 mm (0.059 in.), and alternatively less than 1 mm (0.039 in). The primary package can protect the solid article, so the article is compressed less than 50% during shipping, handling, and storage, alternatively less than 40%, alternatively less than 30%, alternatively less than 20%.

The primary package can protect the solid article from compressing to the point that it has hand dissolution issues (e.g. deforming 1.5 mm and/or 30%) under a compressive force of 50 N (11.24 lbf), alternatively 100 N (22.48 lbf), alternatively 150 N (11.24 lbf), alternatively 175 N (39.34 lbf), alternatively 200 N (45.0 lbf).

The primary package can protect the solid article from compressing to the point that it has hand dissolution issues (e.g. deforming 1.5 mm (0.059 in.) and/or 30%) under a compressive force of at least 5 psi, alternatively at least 10 psi, alternatively at least 15 psi, alternatively at least 18 psi, alternatively at least 20 psi, alternatively at least 25 psi.

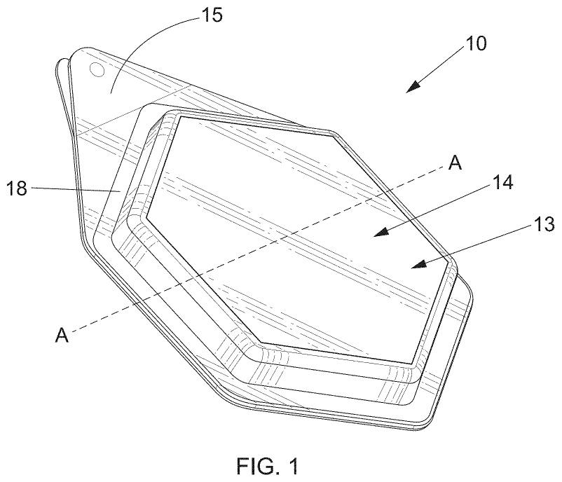

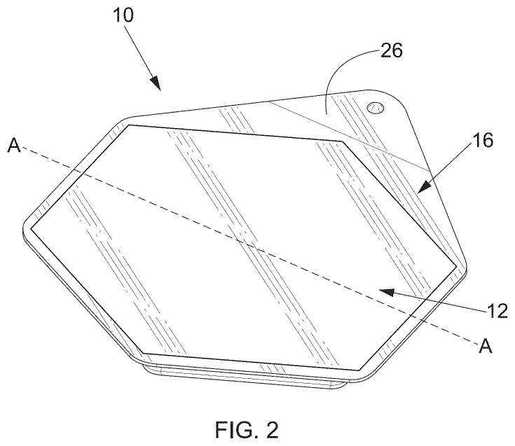

FIGS. 1 and 2 show a first and second perspective view of primary package 10, which is comprised of a flexible lidding film 12 having moisture-barrier properties and is peelably sealed by heat seal 18 to thermoformed portion 13 that includes thermoform compartment 14. Thermoform compartment 14 is configured to receive at least a portion of the solid article, alternatively the entire solid article. The package 10 is designed to contain a solid personal care article, for example a fibrous structure or a porous foam structure that becomes a liquid shampoo, conditioner, or body wash upon hydration.

Lidding film 12 is comprised of any material that can help the primary package withstand about 18 psi to about 25 psi of pressure and provide MVTR of less than 0.5 g/m²/day. The lidding film can be any material that provides the necessary moisture barrier and compression properties to the primary package. In some examples, the lidding film can be a metal containing heat sealable lidding film. For example, the lidding film can be comprised of multilayer laminate comprising the following layers: heat seal coating, metalized film (e.g. 25 μm aluminum foil) or polymer film (e.g. polypropylene film), adhesive that binds the film to polyethylene terephthalate (PET), and 12 μm PET. The lidding film can also contain additional layers including paper, ink, and other coatings.

The thermoformed portion 13 are formed from any suitable material that can help the primary package withstand pressure encountered before use including shipping, handling, and storage and provide MVTR of less than 0.5 g/m²/day. The thermoform portion material can be selected from the group consisting of polypropylene (PP), PET, polyethylene terephthalate glycol (PET-G), poly-chloro-trifluoroethylene/polyethylene terephthalate glycol, and combinations thereof. In some examples, the thermoformed portion may be recyclable and can be selected from the group consisting of PP, PET, and combinations thereof.

The primary package 10 including thermoformed portion 13 and lidding film 12 can be made of any suitable material. The material can be transparent, translucent, opaque, or combinations thereof. In one example, the thermoformed portion 13 can be translucent or transparent and the lidding film 14 can be opaque.

The primary package can be any shape that provides the minimum pressure and moisture barrier properties. The shape of the primary package is a balance between having a small volume, which provides better moisture barrier and compression properties, and a large surface area, which provides better dissolution for the article.

The primary package 10 including the thermoform and lidding film can be a circle, oval, and polygon including parallelogram, rectangle, square, trapezoid, rhombus, pentagon, hexagon, heptagon, hexagon. In one example, primary package 10 including the thermoform portion and lidding film, can be formed in the shape of a hexagon, particularly a regular hexagon.

The perimeter of the lidding film 12 can define pull tab 26. The pull tab 26 may not be sealed to the thermoform portion 13. In one example, pull tab 26 can be devoid of permanent or pressure-sensitive adhesive. In another example, pull tab 26 includes adhesive, but the adhesive in this section has not been exposed to a heat sealer and is therefore not connected to the thermoform portion. The consumer can grab grip portion 15, for example between their thumb and forefinger, and pull the pull tab 26 away from primary package 10 thereby exposing the upper opening of the thermoform compartment and the solid article. In one example, when the consumer pulls pull tab 26, they break heat seal 18 to expose the opening of the thermoform compartment and the solid article. In another example, the primary package cannot be re-closed once it is opened with the pull tab. The pull tab can be knurled or otherwise textured and/or can be made tacky, to help with the grip, since consumers may find it difficult to grip and pull the pull tab and primary package, since he/she is generally using the product with wet, soapy hands in a shower. Pull tab 26 can be any size, preferably large enough so the user can grab and pull the pull tab 26 with his/her thumb and forefinger.

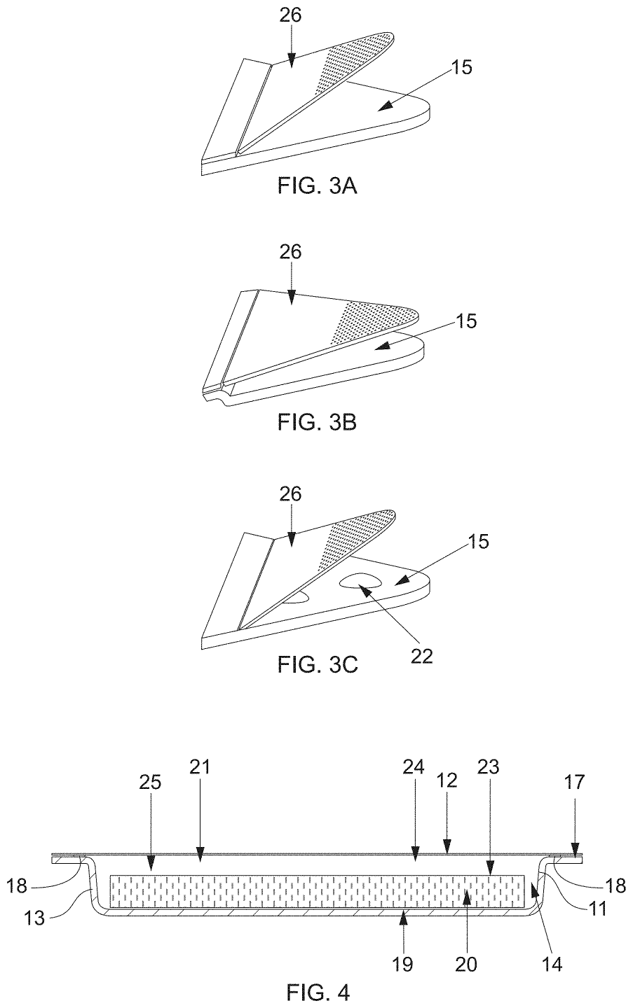

The thermoform portion can be approximately the same shape as the lidding film that includes the pull tab disposed at a corner of the lidding film. Thermoform portion can include grip portion 15, which can be adjacent to heat seal 18. Grip portion 15 can be textured and/or can be made tacky. Grip portion 15 can be in plane with the peripheral flange, as shown in FIG. 3A. Alternatively, grip portion 15 can be stepped out of plane, as in FIG. 3B to help the consumer grip the primary package and separate the lidding film 16 from the grip portion 15. In FIG. 3B, grip portion 15 is stepped out of plane by a transitional plane that has a slope of less than 90 degrees with respect to the flange. In some examples, there can be a raised lip where the flange and transitional plane meet that can further the consumer grip the package and remove the lidding film. Grip portion 15 can have bumps 22 that are raised as compared to grip portion 15 to help the consumer grip the primary package and separate the lidding film from the grip portion 15, as shown in FIG. 3C. Alternatively, the grip portion can have indentations or other textures. In one example, the grip portion can have several ridges, in particular curved ridges, to help the consumer grip the package and lift he pull tab.

In some examples, different products can include different patterns on the thermoform portion including the grip portion and/or lidding film. For instance, if the primary package contains a shampoo solid article, it could contain one circular bump on the grip portion and if the shampoo contains a conditioner solid article, it can have three circular bumps on the grip portion. This could help not only with gripping but could also help people who are visually impaired (or people who shower without wearing corrective lenses) identify the product.

FIG. 4 is a cross-sectional view of the primary package of FIGS. 1 and 2 taken along the plane of the line A-A. FIG. 4 shows thermoformed portion 13 can include thermoform compartment 14 that can be shaped to enclose a solid article 20. Thermoform compartment 14 can have a bottom panel 19, sidewalls 11, and an upper opening 21 which is sealed by the lidding film 12. The thermoformed portion 13 can include peripheral flange 17 which surrounds the package opening and/or thermoform compartment and provides support for lidding film 12. The lidding film 12, can be sealable or laminable by heat or adhesive to peripheral flange 17. The lidding film 12 can include a top and a bottom 24. The solid article 20 includes a top 23 and a bottom.

Headspace 25 is the distance between lidding film bottom 24 and solid article top 23. The headspace can prevent compression of the solid article. The head space can have a height from about 0.25 mm (0.0098 in) to about 2 mm (0.079 in), from about 0.5 mm (0.020 in.) to about 1.5 mm (0.059 in.), alternatively from about 0.7 mm (0.028 in.) to about 1.2 mm (0.047 in.). The headspace height can be from about 5% to about 50% of the height of the thermoform compartment, alternatively from about 5% to about 40%, alternatively from about 5% to about 30%, alternatively from about 6% to about 25%, alternatively from about 7% to about 20%, alternatively from about 10% to about 18%, and alternatively from about 12% to about 17%.

The thermoform can have a sidewall with a thickness from about 0.020 in. to about 0.080 in., alternatively from about 0.020 in. to about 0.060 in., alternatively from about 0.020 in. to about 0.040 in. Alternatively, the thermoformed portion can have a sidewall with a thickness from about 0.01 inch to about 0.1 inch, alternatively about 0.02 inch to about 0.05 inch, and alternatively from about 0.02 inch to about 0.03 inch. In one example, the sidewall can comprise PET or PP.

The thermoform compartment can have a bottom panel with a surface area from about 1.5 in² to about 6 in², alternatively from about 2.5 in² to about 5 in², and alternatively from about 2.25 in² to about 4.5 in².

The thermoform compartment can have a volume from about 0.2 in³ to about 2 in³, alternatively from about 0.3 in³ to about 1 in³, and alternatively from about 0.4 in³ to about 0.7 in³.

The volume of the thermoform compartment can be from about 5% to about 30% larger than the volume of the solid article, alternatively from about 7% to about 25% larger, alternatively from about 10% to about 25% larger, alternatively from about 12% to about 24% larger, alternatively from about 14% to about 22% larger.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Where amount ranges are given, these are to be understood as being the total amount of said ingredient in the composition, or where more than one species fall within the scope of the ingredient definition, the total amount of all ingredients fitting that definition, in the composition. For example, if the composition comprises from 1% to 5% fatty alcohol, then a composition comprising 2% stearyl alcohol and 1% cetyl alcohol and no other fatty alcohol, would fall within this scope.

### Solid Articles

Described herein solid article that can comprise one or more fibrous structures described herein. The personal care product can further comprise a communication directing a consumer to dissolve the structure and/or article and apply the dissolved mixture to hair, hair follicles, skin including the scalp, to achieve a benefit to the target consumer substrate, a rapidly lathering foam, a rapidly rinsing foam, a clean rinsing foam, a conditioning treatment and combinations thereof. The communication may be printed material attached directly or indirectly to the primary packaging or other secondary packaging that contains the fibrous structure or on the fibrous structure itself. Alternatively, the communication may be an electronic or a broadcast message that is associated with the article of manufacture. Alternatively, the communication may describe at least one possible use, capability, distinguishing feature and/or property of the article of manufacture. Alternatively, the communication can contain symbols and or pictures to demonstrate use of the article.

“Dissolvable” means that the Dissolvable Solid Structure is completely soluble in water or it provides a uniform dispersion upon mixing in water according to the Hand Dissolution Test, described hereafter. The Dissolvable Solid Structure can have a hand dissolution value of from about 1 to about 30 strokes, alternatively from about 2 to about 25 strokes, alternatively from about 3 to about 20 strokes, and alternatively from about 4 to about 15 strokes, as measured by the Hand Dissolution Method.

“Fibrous structure” as used herein means a structure that comprises one or more fibrous elements and optionally, one or more particles. In one example, a fibrous structure according to the present invention means an association of fibrous elements and optionally, particles that together form a structure, such as a unitary structure, capable of performing a function.

FIG. 5 is an example of a solid article containing one or more fibrous structures that contain filaments.

The fibrous structures of the present invention may be homogeneous or may be layered. If layered, the fibrous structures may comprise at least two and/or at least three and/or at least four and/or at least five layers. The layer can be fibrous elements, particles, and mixtures thereof.

In one example, the fibrous structure can be a multi-ply fibrous structure that exhibits a basis weight of less than 5000 g/m2 as measured according to the Basis Weight Test Method described herein.

In one example, the fibrous structure of the present invention can be a “unitary fibrous structure.”

“Unitary fibrous structure” as used herein is an arrangement comprising a plurality of two or more and/or three or more fibrous elements that are inter-entangled or otherwise associated with one another to form a fibrous structure. The unitary fibrous structure can optionally contain particles. A unitary fibrous structure of the present invention may be one or more plies within a multi-ply fibrous structure. In one example, a unitary fibrous structure of the present invention may comprise three or more different fibrous elements. In another example, a unitary fibrous structure of the present invention may comprise two different fibrous elements, for example a co-formed fibrous structure, upon which a different fibrous element is deposited to form a fibrous structure comprising three or more different fibrous elements.

“Fibrous element” as used herein means an elongate particulate having a length greatly exceeding its average diameter, i.e. a length to average diameter ratio of at least about 10. A fibrous element may be a filament or a fiber. In one example, the fibrous element can be a single fibrous element rather than a yarn comprising a plurality of fibrous elements.

The fibrous elements of the present invention may be spun from a filament-forming composition also referred to as fibrous element-forming compositions via suitable spinning process operations, such as meltblowing, spunbonding, electro-spinning, and/or rotary spinning.

The fibrous elements of the present invention may be monocomponent and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers. Non-limiting examples of filaments include meltblown and/or spunbond filaments. Non-limiting examples of polymers that can be spun into filaments include natural polymers, such as starch, starch derivatives, cellulose, such as rayon and/or lyocell, and cellulose derivatives, hemicellulose, hemicellulose derivatives, and synthetic polymers including, but not limited to thermoplastic polymer filaments, such as polyesters, nylons, polyolefins such as polypropylene filaments, polyethylene filaments, and biodegradable thermoplastic fibers such as polylactic acid filaments, polyhydroxyalkanoate filaments, polyesteramide filaments and polycaprolactone filaments.

“Fiber” as used herein means an elongate particulate as described above that exhibits a length of less than 5.08 cm (2 in.) and/or less than 3.81 cm (1.5 in.) and/or less than 2.54 cm (1 in.).

Fibers are typically considered discontinuous in nature. Non-limiting examples of fibers include staple fibers produced by spinning a filament or filament tow of the present invention and then cutting the filament or filament tow into segments of less than 5.08 cm (2 in.) thus producing fibers.

In one example, one or more fibers may be formed from a filament of the present invention, such as when the filaments are cut to shorter lengths (such as less than 5.08 cm in length). Thus, in one example, the present invention also includes a fiber made from a filament of the present invention, such as a fiber comprising one or more polymeric structurants and one or more other ingredients, such as surfactants and high melting point fatty materials. Therefore, references to filament and/or filaments of the present invention herein also include fibers made from such filament and/or filaments unless otherwise noted. Fibers are typically considered discontinuous in nature relative to filaments, which are considered continuous in nature.

“Filament-forming composition” and/or “fibrous element-forming composition” as used herein means a composition that can be suitable for making a fibrous element of the present invention such as by meltblowing and/or spunbonding. The filament-forming composition comprises one or more polymeric structurants that exhibit properties that make them suitable for spinning into a fibrous element. In addition, the filament-forming composition may comprise one or more polar solvents, such as water, into which one or more, for example all, of the polymeric structurant and/or one or more, for example all, of surfactants are dissolved and/or dispersed prior to spinning a fibrous element, such as a filament from the filament-forming composition.

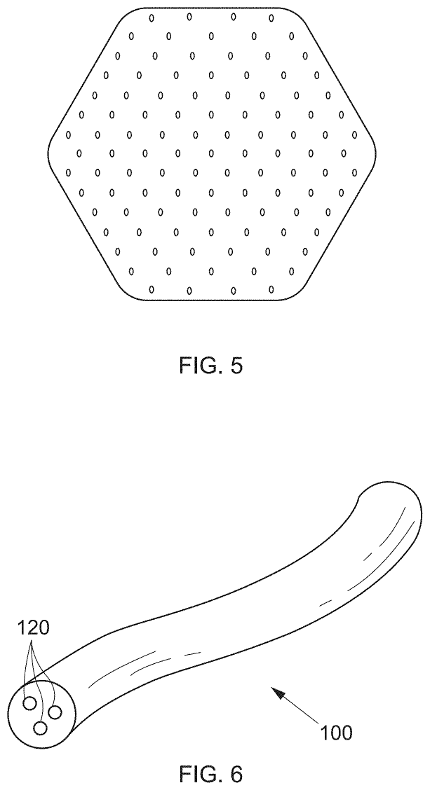

In one example, as shown in FIG. 6, a filament 100 of the present invention made from a filament-forming composition of the present invention is such that one or more additives, for example one or more active agents, may be present in the filament rather than on the filament, such as a coating composition comprising one or more active agents, which may be the same or different from the active agents in the fibrous elements and/or particles.

In one example, one or more additives, such as active agents, may be present in the fibrous element, as shown in FIG. 6 with additives 120 present within filament 100. In another example, one or more additional additives, such as active agents, may be present on a surface of the fibrous element. In another example, a fibrous element of the present invention may comprise one or more additives, such as active agents, that are present in the fibrous element when originally made, but then bloom to a surface of the fibrous element prior to and/or when exposed to conditions of intended use of the fibrous element.

As used herein, “vinyl pyrrolidone copolymer” (and “copolymer” when used in reference thereto) refers to a polymer of the following structure (I):

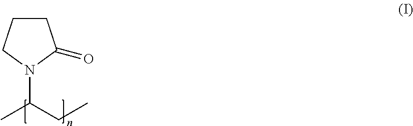

In structure (I), n is an integer such that the polymeric structurant has the degree of polymerization such that it possesses characteristics described herein. For purposes of clarity, the use of the term “copolymer” is intended to convey that the vinyl pyrrolidone monomer can be copolymerized with other non-limiting monomers such as vinyl acetate, alkylated vinyl pyrrolidone, vinyl caprolactam, vinyl valerolactam, vinyl imidazole, acrylic acid, methacrylate, acrylamide, methacrylamide, dimethacrylamide, alkylaminomethacrylate, and alkylaminomethacrylamide monomers.

As used herein, “vinyl acetate-vinyl alcohol copolymer” (and “copolymer” when used in reference thereto) refers to a polymer of the following structure (I):

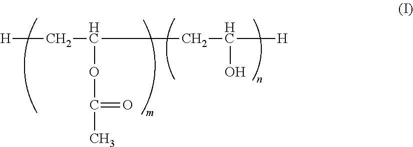

In structure (I), m and n are integers such that the polymeric structurant has the degree of polymerization and percent alcohol characteristics described herein. For purposes of clarity, this use of the term “copolymer” is intended to convey that the partially hydrolyzed polyvinyl acetate of the present invention comprises vinyl alcohol and vinyl acetate units. As discussed below, the polymeric structurant is routinely prepared by polymerizing vinyl acetate monomer followed by hydrolysis of some of the acetate groups to alcohol groups, as opposed to polymerization of vinyl acetate and vinyl alcohol monomer units (due in-part to the instability of vinyl alcohol).

“Conditions of intended use” as used herein means the temperature, physical, chemical, and/or mechanical conditions that a fibrous element and/or particle and/or fibrous structure of the present invention is exposed to when the fibrous element and/or particle and/or fibrous structure is used for one or more of its designed purposes. For instance, if a fibrous element and/or a particle and/or a fibrous structure comprising a fibrous element is designed to be used by a human as a shampoo for hair care purposes, the conditions of intended use will include those temperature, chemical, physical and/or mechanical conditions present during the shampooing of the human's hair. Likewise, if a fibrous element and/or a particle and/or a fibrous structure comprising a fibrous element is designed to be used in a dishwashing operation, by hand or by a dishwashing machine, the conditions of intended use will include the temperature, chemical, physical and/or mechanical conditions present in a dishwashing water and/or dishwashing machine, during the dishwashing operation.

“Active agent” as used herein means an additive that produces an intended effect in an environment external to a fibrous element and/or a particle and/or a fibrous structure comprising a fibrous element of the present invention, such as when the fibrous element and/or a particle and/or fibrous structure is exposed to conditions of intended use of the fibrous element and/or a particle and/or a fibrous structure comprising a fibrous element. In one example, an active agent comprises an additive that treats a surface, including a soft surface (i.e., hair, skin). In another example, an active agent comprises an additive that creates a chemical reaction (i.e., foaming, fizzing, coloring, warming, cooling, lathering, disinfecting and/or clarifying and/or chlorinating, such as in clarifying water and/or disinfecting water and/or chlorinating water). In yet another example, an active agent comprises an additive that treats an environment (i.e., deodorizes, purifies, perfumes). In one example, the active agent is formed in situ, such as during the formation of the fibrous element and/or particle containing the active agent, for example the fibrous element and/or particle may comprise a water-soluble polymer (e.g., starch) and/or a surfactant (e.g., anionic surfactant), which may create a polymer complex or coacervate that functions as the active agent used to treat the hair and/or scalp.

“Treats” as used herein with respect to treating a surface means that the active agent provides a benefit to a surface or environment. Treats includes regulating and/or immediately improving a surface's, cleanliness, smell, purity and/or feel. In one example treating in reference to treating a keratinous tissue (for example skin and/or hair) surface means regulating and/or immediately improving the keratinous tissue's cosmetic appearance and/or feel. For instance, “regulating skin, hair, or nail (keratinous tissue) condition” includes: thickening of skin, hair, or nails (e.g, building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

“Weight ratio” as used herein means the ratio between two materials on their dry basis.

“Water-soluble material” as used herein means a material that is miscible in water. In other words, a material that is capable of forming a stable (does not separate for greater than 5 minutes after forming the homogeneous solution) homogeneous solution with water at ambient conditions.

“Water-insoluble” as used herein is meant that the material, particle, and/or substrate does not dissolve in or readily break apart upon immersion in water. In some instances, water-insoluble materials swell when exposed to water.

“Ambient conditions” as used herein means 23° C.±1.0° C. and a relative humidity of 50% 2%.

As used herein, “molecular weight” or “M.Wt.” refers to the weight average molecular weight unless otherwise stated. Molecular weight is measured using industry standard method, gel permeation chromatography (“GPC”).

“Length” as used herein, with respect to a fibrous element, means the length along the longest axis of the fibrous element from one terminus to the other terminus. If a fibrous element has a kink, curl or curves in it, then the length is the length along the entire path of the fibrous element from one terminus to the other terminus.

“Diameter” as used herein, with respect to a fibrous element, is measured according to the Diameter Test Method described herein. In one example, a fibrous element of the present invention exhibits a diameter of less than 100 μm and/or less than 75 μm and/or less than 50 μm and/or less than 25 μm and/or less than 20 μm and/or less than 15 μm and/or less than 10 μm and/or less than 6 μm and/or greater than 1 μm and/or greater than 3 μm.

“Triggering condition” as used herein in one example means anything, as an act or event, that serves as a stimulus and initiates or precipitates a change in the fibrous element and/or particle and/or fibrous structure of the present invention, such as a loss or altering of the fibrous element's and/or fibrous structure's physical structure and/or a release of an additive, such as an active agent therefrom. In another example, the triggering condition may be present in an environment, such as water, when a fibrous element and/or particle and/or fibrous structure of the present invention is added to the water. In other words, nothing changes in the water except for the fact that the fibrous element and/or fibrous structure of the present invention is added to the water.

“Morphology changes” as used herein with respect to a fibrous element's and/or particle's morphology changing means that the fibrous element experiences a change in its physical structure. Non-limiting examples of morphology changes for a fibrous element and/or particle of the present invention include dissolution, melting, swelling, shrinking, breaking into pieces, exploding, lengthening, shortening, and combinations thereof. The fibrous elements and/or particles of the present invention may completely or substantially lose their fibrous element or particle physical structure or they may have their morphology changed or they may retain or substantially retain their fibrous element or particle physical structure as they are exposed to conditions of intended use.

“By weight on a dry fibrous element basis” and/or “by weight on a dry fibrous structure basis” means the weight of the fibrous element and/or particle and/or fibrous structure, respectively, measured immediately after the fibrous element and/or particle and/or fibrous structure, respectively, has been conditioned in a conditioned room at a temperature of 23° C.±1.0° C. and a relative humidity of 50%±10% for 2 hours. In one example, by weight on a dry fibrous element basis and/or dry fibrous structure basis means that the fibrous element and/or particle and/or fibrous structure comprises less than 20% and/or less than 15% and/or less than 10% and/or less than 7% and/or less than 5% and/or less than 3% and/or to 0% and/or to greater than 0% based on the dry weight of the fibrous element and/or particle and/or fibrous structure of moisture, such as water, for example free water, as measured according to the Water Content Test Method described herein.

“Total level” as used herein, for example with respect to the total level of one or more active agents present in the fibrous element and/or particle and/or fibrous structure, means the sum of the weights or weight percent of all of the subject materials, for example active agents. In other words, a fibrous element and/or particle and/or fibrous structure may comprise 25% by weight on a dry fibrous element basis and/or dry fibrous structure basis of an anionic surfactant, 15% by weight on a dry fibrous element basis and/or dry fibrous structure basis of a nonionic surfactant, 10% by weight of a chelant on a dry fibrous element basis and/or dry fibrous structure basis, and 5% by weight of a perfume a dry fibrous element basis and/or dry fibrous structure basis so that the total level of active agents present in the fibrous element and/or particle and/or fibrous structure is greater than 50%; namely 55% by weight on a dry fibrous element basis and/or dry fibrous structure basis.

“Fibrous structure product” as used herein means a solid form, for example a rectangular solid, sometimes referred to as a sheet, that comprises one or more active agents, for example a fabric care active agent, a dishwashing active agent, a hard surface active agent, and mixtures thereof. In one example, a fibrous structure product of the present invention comprises one or more surfactants, one or more enzymes (such as in the form of an enzyme prill), one or more perfumes and/or one or more suds suppressors. In another example, a fibrous structure product of the present invention comprises a builder and/or a chelating agent. In another example, a fibrous structure product of the present invention comprises a bleaching agent (such as an encapsulated bleaching agent).

“Associate,” “Associated,” “Association,” and/or “Associating” as used herein with respect to fibrous elements and/or particle means combining, either in direct contact or in indirect contact, fibrous elements and/or particles such that a fibrous structure is formed. In one example, the associated fibrous elements and/or particles may be bonded together for example by adhesives and/or thermal bonds. In another example, the fibrous elements and/or particles may be associated with one another by being deposited onto the same fibrous structure making belt and/or patterned belt.

“Ply” or “Plies” as used herein means an individual fibrous structure optionally to be disposed in a substantially contiguous, face-to-face relationship with other plies, forming a multiple ply fibrous structure. It is also contemplated that a single fibrous structure can effectively form two “plies” or multiple “plies”, for example, by being folded on itself.

As used herein, the articles “a” and “an” when used herein, for example, “an anionic surfactant” or “a fiber” is understood to mean one or more of the material that is claimed or described.

As used herein, the terms “include,” “includes,” and “including,” are meant to be non-limiting.

### Fibrous Structure

The fibrous structure of the present invention can comprise a plurality of fibrous elements, for example a plurality of filaments. Non-limiting examples of products that can utilize the fibrous structure include hand cleansing substrates, hair shampoo, hair conditioner or other hair treatment substrates including combination hair shampoo and conditioning substrates, body cleansing substrates, shaving preparation substrates, personal care substrates containing pharmaceutical or other skin care active, moisturizing substrates, sunscreen substrates, chronic skin benefit agent substrates (e.g., vitamin-containing substrates, alpha-hydroxy acid-containing substrates, etc.), deodorizing substrates, fragrance-containing substrates, and so forth.

FIG. 5 shows a fibrous article according to the present invention. The fibrous article comprises one or more fibrous structures comprising a plurality of fibrous elements, in this case filaments. The filaments can contain the polymeric structurant and the surfactant. In some examples, the filaments can also comprise other ingredients including a high melting point fatty amphiphiles and/or cationic surfactants and/or anionic surfactants and/or oil soluble acid. The polymeric structurant, surfactant, and other ingredients, if present, can be dispersed throughout the filament. The filament can be homogenous. In some examples, other ingredients, like the oil soluble acid can be present in a coating or an agglomerate. In other examples, the oil soluble acid may not be present in particles or agglomerates that are incorporated into or sprayed onto of the fibrous structure.

The fibrous article can be used as a hair conditioner and can include: fibrous elements containing (a) from about 1 wt. % to about 50 wt % polymeric structurant; (b) from about 10 wt. % to about 85 wt. % of a high melting point fatty material such as a fatty amphiphile, (c) from about 1 wt. % to about 60 wt. % of a cationic surfactant; and (d) from about 0.1-10% an oil soluble acid. When water is added to the fibrous structure at a ratio of about 10:1 a lamellar structure can be formed.

The fibrous article can be used as a hair shampoo and can include: fibrous elements containing (a) from about 1 wt. % to about 50 wt % polymeric structurant; (b) from about 10 wt. % to about 90 wt. % of a surfactant system comprising a surfactant selected from the group consisting of anionic, cationic, zwitterionic surfactant, and combinations thereof; and (c) optionally from about 0.1 wt. % to about 2 wt. % high molecular weight cationic surfactant.

In one example, the fibrous structure comprises a plurality of identical or substantially identical from a compositional perspective of fibrous elements according to the present invention. In another example, the fibrous structure may comprise two or more different fibrous elements according to the present invention.

In another example, the fibrous structure may exhibit different regions, such as different regions of basis weight, density and/or caliper. In yet another example, the fibrous structure may comprise texture on one or more of its surfaces. A surface of the fibrous structure may comprise a pattern, such as a non-random, repeating pattern. The fibrous structure may be embossed with an emboss pattern. In another example, the fibrous structure may comprise apertures. The apertures may be arranged in a non-random, repeating pattern.

The fibrous structure of the present invention may be used as is or may be coated with one or more active agents.

In one example, the fibrous structure of the present invention exhibits a thickness of greater than 0.01 mm and/or greater than 0.05 mm and/or greater than 0.1 mm and/or to about 100 mm and/or to about 50 mm and/or to about 20 mm and/or to about 10 mm and/or to about 5 mm and/or to about 2 mm and/or to about 0.5 mm and/or to about 0.3 mm as measured by the Thickness Test Method described herein.

For fibrous structures, the structure can comprise a significant number of dissolvable fibers with an average diameter less than about 150 micron, alternatively less than about 100 micron, alternatively less than about 10 micron, and alternatively less than about 1 micron with a relative standard deviation of less than 100%, alternatively less than 80%, alternatively less than 60%, alternatively less than 50%, such as in the range of 10% to 50%, for example. As set forth herein, the significant number means at least 10% of all the dissolvable fibers, alternatively at least 25% of all the dissolvable fibers, alternatively at least 50% of all the dissolvable fibers, alternatively at least 75% of all the dissolvable fibers. The significant number may be at least 99% of all the dissolvable fibers. Alternatively, from about 50% to about 100% of all the dissolvable fibers may have an average diameter less than about 10 micron. The dissolvable fibers produced by the method of the present disclosure can have a significant number of dissolvable fibers with an average diameter less than about 1 micron, or sub-micron fibers. In an embodiment, fibrous structure may have from about 25% to about 100% of all the dissolvable fibers with an average diameter less than about 1 micron, alternatively from about 35% to about 100% of all the dissolvable fibers with an average diameter less than about 1 micron, alternatively from about 50% to about 100% of all the dissolvable fibers with an average diameter less than about 1 micron, and alternatively from about 75% to about 100% of all the dissolvable fibers with an average diameter less than about 1 micron.

The structure can be characterized in one aspect by its Specific Surface Area. The structure can have a Specific Surface Area of from about 0.03 m²/g to about 0.25 m²/g, alternatively from about 0.035 m²/g to about 0.22 m²/g, alternatively from about 0.04 m²/g to about 0.19 m²/g, and alternatively from about 0.045 m²/g to about 0.16 m²/g.

The structure can be a flat, flexible structure in the form of a pad, a strip, or tape and having a thickness of from about 0.5 mm to about 10 mm, alternatively from about 1 mm to about 9 mm, alternatively from about 2 mm to about 8 mm, and alternatively from about 3 mm to about 7 mm as measured by the below methodology. The Structure can be a sheet having a thickness from about 5 mm to about 6.5 mm. Alternatively, two or more sheets are combined to form a Structure with a thickness of about 5 mm to about 10 mm.

The structure can have a basis weight of from about 200 grams/m² to about 2,000 grams/m², alternatively from about 400 g/m² to about 1,200 g/m², alternatively from about 600 g/m² to about 2,000 g/m², and alternatively from about 700 g/m² to about 1,500 g/m².

The structure can have a dry density of from about 0.08 g/cm³ to about 0.40 g/cm³, alternatively from about 0.08 g/cm³ to about 0.38 g/cm³, alternatively from about 0.10 g/cm³ to about 0.25 g/cm³, and alternatively from about 0.12 g/cm³ to about 0.20 g/cm³.

Non-limiting examples of other fibrous structures suitable for the present invention are disclosed in U.S. Pat. Nos. 8,980,816 and 9,139,802 and U.S. Pub. No. 2013/0171421 are hereby incorporated by reference.

### Fibrous Elements

The fibrous element, such as a filament and/or fiber, of the present invention comprises one or more polymeric structurants. In addition to the polymeric structurants, the fibrous element may further comprise one or more high melting point fatty materials, one or more cationic surfactants, one or more oil soluble acids and optional ingredients.

### Polymeric Structurant

To improve the fiber spinning of low viscosity material, such as molten fatty alcohols, fatty quaternary ammonium compounds, fatty acids, etc., a polymeric ingredient called a structurant can be added. The structurant increases the shear and extensional viscosity of the fluid to enable fiber formation.

The structurant can be included at a level of from about 1 wt. % to about 50 wt. %, alternatively from about 1 wt. % to about 30 wt. %, alternatively from about 1 wt. % to about 10 wt. %, alternatively from about 2 wt. % to about 6 wt. %, and alternatively from about 3 wt. % to about 5 wt. % of the composition. The structurant has a weight average molecular weight of from about 10,000 to about 6,000,000 g/mol. A balance can be struck between concentration and molecular weight, such that when a lower molecular weight species is used, it requires a higher level to result in optimal fiber spinning. Likewise, when a higher molecular species is used, lower levels can be used to achieve optimal fiber spinning. The one or more structurants can be selected such that their weight average molecular weight is from about 15,000 g/mol to about 500,000 g/mol, alternatively from about 20,000 g/mol to about 500,000 g/mol, alternatively from about 50,000 g/mol to about 400,000 g/mol, alternatively from about 60,000 g/mol to about 300,000 g/mol, and alternatively from about 70,000 g/mol to about 200,000 g/mol.

The polymeric structurant can include, but are not limited to, synthetic polymers as described in US Pub. No. US 2010/0173817 including polymers derived from acrylic monomers such as the ethylenically unsaturated carboxylic monomers and ethylenically unsaturated monomers as described in U.S. Pat. No. 5,582,786 and EP-A-397410. The polymeric structurant which are suitable may also be selected from naturally sourced polymers including those of plant origin examples which are described in US Pub. No. US 2010/0173817. Modified natural polymers are also useful as polymeric structurant and are included in US Pub. No. US 2010/0173817. In one embodiment, water-soluble polymers include polyvinyl alcohols, polyacrylates, polymethacrylates, copolymers of acrylic acid and methyl acrylate, polyvinylpyrrolidones, polyvinylmethylether, polyvinylformamide, polyacrylamide, polyalkylene oxides, starch and starch derivatives, pullulan, gelatin, hydroxypropylmethylcelluloses, methycelluloses, and carboxymethycelluloses, salts and combinations thereof. In another embodiment, water-soluble polymers include polyvinyl alcohols, and hydroxypropylmethylcelluloses. Suitable polyvinyl alcohols include those available from Celanese Corporation (Dallas, Tex.) under the CELVOL® trade name. Suitable hydroxypropylmethylcelluloses include those available from the Dow Chemical Company (Midland, Mich.) under the METHOCEL® trade name.

The above mentioned polymeric structurant may be blended with any single starch or combination of starches as a filler material in such an amount as to reduce the overall level of water-soluble polymers needed, so long as it helps provide the personal care Dissolvable Solid Structure with the requisite structure and physical/chemical characteristics as described herein.

In such instances, the combined weight percentage of the polymeric structurant and starch-based material generally ranges from about 10% to about 75%, alternatively from about 15% to about 40%, and alternatively from about 20% to about 30% by weight relative to the total weight of the Dissolvable Solid Structure. The weight ratio of the polymeric structurant to the starch-based material can generally range from about 1:10 to about 10:1, alternatively from about 1:8 to about 8:1, alternatively from about 1:7 to about 7:1, and alternatively from about 6:1 to about 1:6.

Typical sources for starch-based materials can include cereals, tubers, roots, legumes and fruits. Native sources can include corn, pea, potato, banana, barley, wheat, rice, sago, amaranth, tapioca, arrowroot, canna, sorghum, and waxy or high amylase varieties thereof. The starch-based materials may also include native starches that are modified using any modification known in the art, including those described in U.S. Ser. No. 61/120,786.

The one or more fibrous-element forming polymeric structurants can comprise one or more polyvinyl alcohols. The one or more polyvinyl alcohols may exhibit a weight average molecular weight of from about 10,000 g/mol to about 40,000,000 g/mol, preferably from about 20,000 g/mol to about 30,000,000 g/mol, more preferably from about 35,000 g/mol to about 20,000,000 g/mol, even more preferably from about 40,000 g/mol to about 5,000,000 g/mol, most preferably from about 40,000 g/mol to about 500,000 g/mol.

The one or more fibrous-element forming polymeric structurant materials may comprise two or more polyvinyl alcohols. One of the two or more polyvinyl alcohols may exhibit a weight average molecular weight of from about 10,000 g/mol to about 100,000 g/mol, preferably from about 20,000 g/mol to about 50,000 g/mol, more preferably from about 25,000 g/mol to about 45,000 g/mol, and the other of two or more polyvinyl alcohols may exhibit a weight average molecular weight of from about 105,000 g/mol to about 40,000,000 g/mol, preferably from about 110,000 g/mol to about 20,000,000 g/mol, more preferably from about 120,000 g/mol to about 500,000 g/mol.

Non-limiting examples of fibrous-element forming polymeric structurant include water-soluble hydroxyl polymers, water-soluble thermoplastic polymers, water-soluble biodegradable polymers, water-soluble non-biodegradable polymers and mixtures thereof.

The one or more fibrous-element forming polymeric structurant materials may further comprise starch. Preferably, the one or more fibrous-element forming polymeric structurant materials may comprise one or more polyvinyl alcohols and starch.

The one or more fibrous-element forming materials may further comprise carboxymethyl cellulose. The one or more fibrous-element forming polymeric structurant materials may comprise one or more polyvinyl alcohols and carboxymethyl cellulose.

The structurant can be soluble in an oily mixture to enable viscosity build for fiber spinning. In addition, the structurant should also be soluble in water to promote removal and to prevent buildup. Suitable structurants include, but are not limited to, polyvinylpyrrolidone, polydimethylacrylamides, and combinations thereof. These polymers are oil (fatty alcohol, fatty acid, fatty quaternary ammonium compounds) soluble, water soluble, and capable of being produced at high weight average molecular weights. For example, suitable polymers for use are PVP K120 from Ashland Inc., having a weight average molecular weight of about 3,500,000 g/mol is soluble in the oil and water and enables fibers to be formed and collected onto a belt. Additional suitable polymers include copolymers of polyvinylpyrrolidone, such as Ganex® or PVP/VA (weight average molecular weight of about 50,000 g/mol) copolymers from Ashland Inc., also performed as suitable structurants but a higher level was utilized to be effective due to their lower weight average molecular weight. In addition, copolymers of polydimethylacrylamide also function as a suitable structurant. Hydroxyl propyl cellulose can also function as a suitable structurant.

### Dispersing Agents

The fibrous elements, it has been found that the addition of a dispersing agent can greatly increase the wetting, hydration, and dispersion of the conditioner materials. The dispersing agent can be included at a level of from about 1 wt. % to about 30 wt. % of the composition, alternatively from about 5 wt. % to about 15 wt. %, and alternatively from about 5 wt. % to about 10 wt %. A surfactant from the nonionic class of alkyl glucamides can improve the wetting and hydration when added to the solid conditioner formula. The alkyl glucamide surfactant contains a hydrophobic tail of about 8-18 carbons and a nonionic head group of glucamide. For glucamide, the presence of the amide and hydroxyl groups may provide sufficient polarity that balances the hydrophobic carbon tail in such a way to permit the surfactant's solubility in the conditioner oils and also imparts a rapid dispersion of the conditioner ingredients upon exposure to water. Other similar dispersing agents include, but are not limited to, reverse alkyl glucamides, cocoamiodpropyl betaines, alkyl glucoside, Triethanol amine, cocamide MEAs and mixtures thereof.

### Surfactants

The fibrous articles can comprise one or more detersive surfactants suitable for application to the hair or skin. Surfactants suitable for use in the article can include anionic surfactants, nonionic surfactants, cationic surfactants, zwitterionic surfactants, amphoteric surfactants, polymeric surfactants or combinations thereof. Although representative surfactants are described herein, the skilled artisan will recognize that other surfactants can be readily substituted and similar benefits can be derived from use of the vinyl acetate-vinyl alcohol copolymers described herein. Each patent described throughout this application is incorporated herein by reference to the extent each provides guidance regarding surfactants suitable for inclusion in the article.

In one embodiment, the article is a lathering dissolvable solid personal care product (dried) and comprises from about 10 wt % to about 75 wt % surfactant, in one embodiment from about 25 wt % to about 70 wt % surfactant, in another embodiment from about 40 wt % to about 65 wt % surfactant.

Suitable anionic surfactants include alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In one embodiment, the anionic surfactant is sodium lauryl sulfate or sodium laureth sulfate.

In one embodiment, the anionic surfactant is at least one branched sulfate having the formula CH₃—(CH₂)_{z}—CH(R¹)—CH₂—O—(CH₂CH(R²)O)_{y}—SO₃M; where z is from about 3 to about 14; IV represents H or a hydrocarbon radical comprising 1 to 4 carbon atoms, R² is H or CH₃; R¹ and R² are not both H; y is 0 to about 7; the average value of y is about 1 when y is not=0; and M is a mono-valent or di-valent, positively-charged cation. Examples of mono-valent positively charged cations include ammonium, sodium, potassium, triethanolamine cation, and examples of di-valent positively charged cations include magnesium. For the foregoing branched sulfates, “average value” means that whereas the composition may comprise molecules having a value of y of other than 1, the average value of y all molecules in the composition is about 1.

Suitable amphoteric or zwitterionic surfactants include those which are known for use in shampoo or other cleansing products. Non limiting examples of suitable zwitterionic or amphoteric surfactants are described in U.S. Pat. Nos. 5,104,646 and 5,106,609, which are incorporated herein by reference in their entirety.

Suitable amphoteric surfactants include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants include cocoamphoacetate, cocoamphodiacetate, lauroamphoacetate, lauroamphodiacetate, and mixtures thereof.

Suitable zwitterionic surfactants include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable nonionic surfactants for use in the present invention include those described in McCutcheon's Detergents and Emulsifiers, North American edition (2010), Allured Publishing Corp., and McCutcheon's Functional Materials, North American edition (2010). Suitable nonionic surfactants for use in the Structure of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

In another embodiment, the nonionic surfactant is selected from sorbitan esters and alkoxylated derivatives of sorbitan esters including sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), sorbitan isostearate, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), all available from Uniqema, and combinations thereof.

Suitable copolymer surfactants include, but are not limited to, block copolymers of ethylene oxide and fatty alkyl residues, block copolymers of ethylene oxide and propylene oxide, hydrophobic ally modified polyacrylates, hydrophobically modified celluloses, silicone polyethers, silicone copolyol esters, diquaternary polydimethylsiloxanes, and co-modified amino/polyether silicones.

The surfactant can be a combination of surfactants wherein one or more surfactants from Group I, wherein Group I comprises anionic surfactants, and one or more surfactants from Group II, wherein Group II comprises a surfactant selected from the group consisting of amphoteric, zwitterionic and combinations thereof; wherein the ratio of Group I to Group II surfactants is from about 90:10 to about 30:70.

### Cationic Surfactant

The fibrous element can contain a cationic surfactant can be included at a level of from about 1 wt. % to about 60 wt. %, alternatively from about 10 wt. % to about 50 wt. %, alternatively from about 20 wt. % to about 40 wt. % of the composition. Cationic surfactant useful herein can be one cationic surfactant or a mixture of two or more cationic surfactants. The cationic surfactant can be selected from the group consisting of, but not limited to: a mono-long alkyl amine, a tertiary amine, and combinations thereof.

The fibrous structure can also contain cationic surfactants including a mono-long alkyl quaternized ammonium salt; a combination of a mono-long alkyl quaternized ammonium salt and a di-long alkyl quaternized ammonium salt; a combination of a mono-long alkyl amine and a di-long alkyl quaternized ammonium salt; and a combination of a mono-long alkyl amine and a mono-long alkyl quaternized ammonium salt, a tertiary amine and combinations thereof. In these examples, the surfactant is quaternized and can form a gel network without the addition of acid. However, it can be advantageous to add the oil soluble acids, as described herein, to help adjust the pH.

### Mono-Long Alkyl Amine

Mono-long alkyl amine useful herein are those having one long alkyl chain of from 12 to 30 carbon atoms, alternatively from 16 to 24 carbon atoms, alternatively from 18 to 22 carbon atoms. Mono-long alkyl amines useful herein also include mono-long alkyl amidoamines Primary, secondary, and tertiary fatty amines are useful.

Tertiary amido amines having an alkyl group of from about 12 to about 22 carbons can be used in the fibrous elements. Exemplary tertiary amido amines include: stearamidopropyl dimethylamine, stearamidopropyl diethylamine, stearamidoethyl diethylamine, stearamidoethyl dimethylamine, palmitamido propyldimethyl amine, palmitamidopropyldiethyl amine, palmitamidoethyldiethyl amine, palmitamidoethyldimethylamine, behenamidopropyldimethylamine, behenamidopropyl diethylamine, behenamidoethyl diethylamine, behenamido ethyldimethylamine, arachid amidopropyl dimethylamine, arachid amidopropyl diethylamine, arachid amidoethyl diethylamine, arachid amidoethyl dimethylamine, diethylaminoethyl stearamide. Useful amines in the present invention are disclosed in U.S. Pat. No. 4,275,055, Nachtigal, et al.

### Oil Soluble Acid

The fibrous elements can contain can contain from about 0.01 wt. % to 10 wt. % oil soluble acid, alternatively from about 0.1 wt. % to about 9 wt. %, alternatively about 0.25 wt. % to about 7 wt. %., alternatively from about 0.3 wt. % to about 5 wt. %.

The acid can be an oil soluble acid. If the acid is not soluble in the melt, then the melt cannot be spun because the melt is not homogenous, which, for example, can the fibrous structures to break during spinning and/or clog the die.

The mono-long alkyl amine can be used in combination with oil soluble acids such as salicylic acid, lactic acid, acetic acid, malic acid, succinic acid, sorbic acid, 2,4-dihydroxybenzoic acid, maleic acid. In one example, the filaments can be substantially free of or free of l-glutamic acid, fumaric acid, tartaric acid, citric acid, l-glutamic hydrochloride, citric acid, and mixtures thereof. A molar ratio of the amine to the acid of from about 1:0.3 to about 1:2, alternatively from about 1:0.4 to about 1:1.

In some examples, the fibrous elements are free of or substantially free of hydrochloric acid, citric acid, and combinations thereof. “Substantially free” of hydrochloric acid, citric acid, and combinations thereof means less than 0.05 wt. %, less than 0.04 wt. %, less than 0.03 wt. %, less than 0.02 wt. %, and/or less than 0.01 wt. %.

### High Melting Point Fatty Material

The fibrous element can contain one or more high melting point fatty materials. The high melting point fatty material can be included at a level of from about 10 wt. % to about 85 wt. %, alternatively from about 20 wt. % to about 70 wt. %, alternatively from about 50 wt. % to about 70 wt. %, alternatively from about 10 wt. % to about 20 wt. % of the composition. The fatty material can be selected from the group consisting of, but not limited to, fatty amphiphiles, fatty alcohol, fatty acid, fatty amide, fatty ester and combinations thereof.

The high melting point fatty material useful herein can have a melting point of 25° C. or higher, alternatively 40° C. or higher, alternatively 45° C. or higher, alternatively 50° C. or higher, in view of stability of the emulsion especially the gel matrix. Such melting point is up to about 90° C., alternatively up to about 80° C., alternatively up to about 70° C., alternatively up to about 65° C., in view of easier manufacturing and easier emulsification. The high melting point fatty material can be used as a single compound or as a blend or mixture of at least two high melting point fatty material. When used as such blend or mixture, the above melting point means the melting point of the blend or mixture.

The high melting point fatty material useful herein can be selected from the group consisting of fatty alcohols, fatty acids, fatty alcohol derivatives, fatty acid derivatives, fatty amides, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound but is done so for convenience of classification and nomenclature. Further, it is understood by the artisan that, depending on the number and position of double bonds, and length and position of the branches, certain compounds having certain required carbon atoms may have a melting point of less than the above. Such compounds of low melting point are not intended to be included in this section. Nonlimiting examples of the high melting point materials are found in International Cosmetic Ingredient Dictionary, Fifth Edition, 1993, and CTFA Cosmetic Ingredient Handbook, Second Edition, 1992.

Among a variety of high melting point fatty materials, fatty alcohols can be used in the composition described herein. The fatty alcohols useful herein are those having from about 14 to about 30 carbon atoms, alternatively from about 16 to about 22 carbon atoms. These fatty alcohols are saturated and can be straight or branched chain alcohols.

Suitable fatty alcohols include, but are not limited to, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

Generally, in the mixture, the weight ratio of cetyl alcohol to stearyl alcohol is from about 1:9 to 9:1, alternatively from about 1:4 to about 4:1, alternatively from about 1:2.3 to about 1.5:1.

When using higher level of total cationic surfactant and high melting point fatty materials, the mixture has the weight ratio of cetyl alcohol to stearyl alcohol of from about 1:1 to about 4:1, alternatively from about 1:1 to about 2:1, alternatively from about 1.2:1 to about 2:1, in view of maintaining acceptable consumer usage. It may also provide more conditioning on damaged part of the hair.

### Extensional Aids

The fibrous elements can contain extensional aids. Non-limiting examples of extensional aids can include polymers, other extensional aids, and combinations thereof.

In one example, the extensional aids have a weight-average molecular weight of at least about 500,000 Da. The weight average molecular weight of the extensional aid is from about 500,000 Da to about 25,000,000 Da, alternatively from about 800,000 Da to about 22,000,000 Da, alternatively from about 1,000,000 Da to about 20,000,000 Da, and alternativley from about 2,000,000 Da to about 15,000,000 Da. The high molecular weight extensional aids are preferred in some examples of the invention due to the ability to increase extensional melt viscosity and reducing melt fracture.

The extensional aid, when used in a meltblowing process, can be added to the composition of the present invention in an amount effective to visibly reduce the melt fracture and capillary breakage of fibers during the spinning process such that substantially continuous fibers having relatively consistent diameter can be melt spun. Regardless of the process employed to produce fibrous elements and/or particles, the extensional aids, when used, can be present from about 0.001% to about 10%, by weight on a dry fibrous element basis and/or dry fibrous structure basis, in one example, and in another example from about 0.005 to about 5%, by weight on a dry fibrous element basis and/or dry fibrous structure basis, in yet another example from about 0.01 to about 1%, by weight on a dry fibrous element basis and/or dry fibrous structure basis, and in another example from about 0.05% to about 0.5%, by weight on a dry fibrous element basis and/or dry fibrous structure basis.

Non-limiting examples of polymers that can be used as extensional aids can include alginates, carrageenans, pectin, chitin, guar gum, xanthum gum, agar, gum arabic, karaya gum, tragacanth gum, locust bean gum, alkylcellulose, hydroxyalkylcellulose, carboxyalkylcellulose, and mixtures thereof.

Nonlimiting examples of other extensional aids can include modified and unmodified polyacrylamide, polyacrylic acid, polymethacrylic acid, polyvinyl alcohol, polyvinylacetate, polyvinylpyrrolidone, polyethylene vinyl acetate, polyethyleneimine, polyamides, polyalkylene oxides including polyethylene oxide, polypropylene oxide, polyethylenepropylene oxide, and mixtures thereof.

### Optional Ingredients

The structure optionally comprises from about 1 wt. % to about 25 wt. % plasticizer, in one embodiment from about 3 wt % to about 20 wt. % plasticizer, in one embodiment from about 5 wt. % to about 15 wt. % plasticizer.

When present in the structures, non-limiting examples of suitable plasticizing agents include polyols, copolyols, polycarboxylic acids, polyesters and dimethicone copolyols.

Examples of useful polyols include, but are not limited to, glycerin, diglycerin, propylene glycol, ethylene glycol, butylene glycol, pentylene glycol, cyclohexane dimethanol, hexane diol, polyethylene glycol (200-600), sugar alcohols such as sorbitol, manitol, lactitol, isosorbide, glucamine, N-methylglucamine and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Other suitable plasticizers include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water soluble plasticizer known to one skilled in the art of the foods and plastics industries; and mixtures thereof.

EP 0283165 B1 discloses suitable plasticizers, including glycerol derivatives such as propoxylated glycerol.

The Structure may comprise other optional ingredients that are known for use or otherwise useful in compositions, provided that such optional materials are compatible with the selected essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1992.

Emulsifiers suitable as an optional ingredient herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Further non-limiting examples of such optional ingredients include preservatives, perfumes or fragrances, coloring agents or dyes, conditioning agents, hair bleaching agents, thickeners, moisturizers, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, hair colorants, hair perming agents, pigment particles, anti-acne agents, anti-microbial agents, sunscreens, tanning agents, exfoliation particles, hair growth or restorer agents, insect repellents, shaving lotion agents, co-solvents or other additional solvents, and similar other materials. Further non-limiting examples of optional ingredients include encapsulated perfumes, such as by β-cyclodetrins, polymer microcapsules, starch encapsulated accords and combinations thereof.

Suitable conditioning agents include high melting point fatty materials, silicone conditioning agents and cationic conditioning polymers. Suitable materials are discussed in US 2008/0019935, US 2008/0242584 and US 2006/0217288.

### Methods of Use

The compositions described herein may be used for cleaning, condition, and/or treating hair, hair follicles, and/or skin including the scalp. The method for treating these consumer substrates may comprise the steps of: a) applying an effective amount of the structure to the hand, b) wetting the structure with water to dissolve the solid, c) applying the dissolved material to the target consumer substrate such as to clean, condition, or treat it, and d) rinsing the diluted treatment composition from the consumer substrate. These steps can be repeated as many times as desired to achieve the desired cleansing and or treatment benefit. When the structure is a conditioner, it can be applied before and/or after and/or concurrently with a shampoo.

A method useful for providing a benefit to hair, hair follicles, and/or skin including the scalp, includes the step of applying a composition according to the first embodiment to these target consumer substrates in need of regulating.

Alternatively, a useful method for regulating the condition of hair, hair follicles, skin, and/or skin including the scalp, includes the step of applying one or more compositions described herein to these target consumer substrates in need of regulation.

The amount of the composition applied, the frequency of application and the period of use will vary widely depending upon the purpose of application, the level of components of a given composition and the level of regulation desired. For example, when the composition is applied for whole body or hair treatment, effective amounts generally range from about 0.5 grams to about 10 grams, alternatively from about 1.0 grams to about 5 grams, and alternatively from about 1.5 grams to about 3 grams.

### Exposure to Triggering Condition

The conditioning ingredients, including the cationic surfactant and fatty alcohol, may be released from the fibrous element and/or fibrous structure when the fibrous element and/or fibrous structure is exposed to a triggering condition. In one example, one or more active agents may be released from the fibrous element and/or fibrous structure or a part thereof when the fibrous element and/or fibrous structure or the part thereof loses its identity, in other words, loses its physical structure. For example, a fibrous element and/or fibrous structure loses its physical structure when the polymeric structurant dissolves, melts or undergoes some other transformative step such that its structure is lost. In one example, the one or more active agents are released from the fibrous element and/or fibrous structure when the fibrous element's and/or fibrous structure's morphology changes.

In another example, one or more active agents may be released from the fibrous element and/or fibrous structure or a part thereof when the fibrous element and/or fibrous structure or the part thereof alters its identity, in other words, alters its physical structure rather than loses its physical structure. For example, a fibrous element and/or fibrous structure alters its physical structure when the polymeric structurant swells, shrinks, lengthens, and/or shortens, but retains its filament-forming properties.

In another example, one or more active agents may be released from the fibrous element and/or fibrous structure with its morphology not changing (not losing or altering its physical structure).

In one example, the fibrous element and/or fibrous structure may release an active agent upon the fibrous element and/or fibrous structure being exposed to a triggering condition that results in the release of the active agent, such as by causing the fibrous element and/or fibrous structure to lose or alter its identity as discussed above. Non-limiting examples of triggering conditions include exposing the fibrous element and/or fibrous structure to solvent, a polar solvent, such as alcohol and/or water, and/or a non-polar solvent, which may be sequential, depending upon whether the filament-forming composition comprises a polar solvent-soluble material and/or a non-polar solvent-soluble material; exposing the fibrous element and/or particle and/or fibrous structure to heat, such as to a temperature of greater than 75° F. and/or greater than 100° F. and/or greater than 150° F. and/or greater than 200° F. and/or greater than 212° F.; exposing the fibrous element and/or particle and/or fibrous structure to cold, such as to a temperature of less than 40° F. and/or less than 32° F. and/or less than 0° F.; exposing the fibrous element and/or fibrous structure to a force, such as a stretching force applied by a consumer using the fibrous element and/or fibrous structure; and/or exposing the fibrous element and/or fibrous structure to a chemical reaction; exposing the fibrous element and/or fibrous structure to a condition that results in a phase change; exposing the fibrous element and/or fibrous structure to a pH change and/or a pressure change and/or temperature change; exposing the fibrous element and/or fibrous structure to one or more chemicals that result in the fibrous element and/or fibrous structure releasing one or more of its active agents; exposing the fibrous element and/or particle and/or fibrous structure to ultrasonics; exposing the fibrous element and/or fibrous structure to light and/or certain wavelengths; exposing the fibrous element and/or fibrous structure to a different ionic strength; and/or exposing the fibrous element and/or fibrous structure to an active agent released from another fibrous element and/or fibrous structure.

In one example, one or more active agents may be released from the fibrous elements of the present invention when a fibrous structure product comprising the fibrous elements is subjected to a triggering step such as forming a wash liquor by contacting the fibrous structure product with water.

### Method for Making Fibrous Elements and Structures

The fibrous elements of the present invention may be made by any suitable process. A non-limiting example of a suitable process for making the fibrous elements is described below.

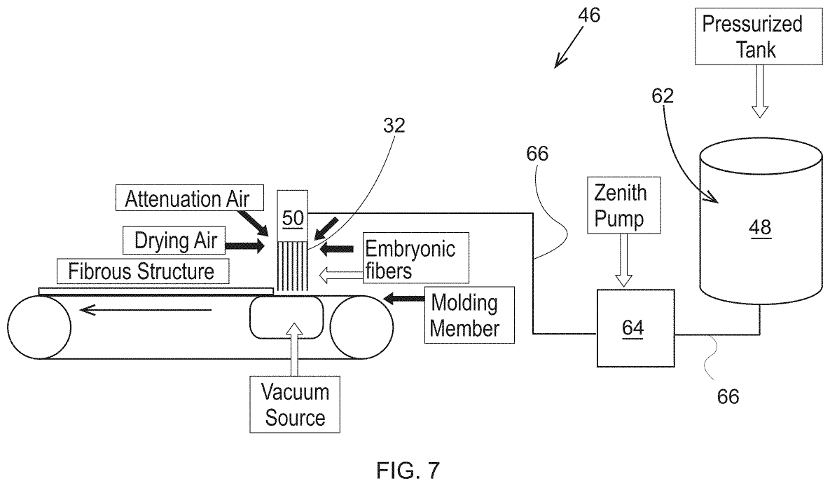

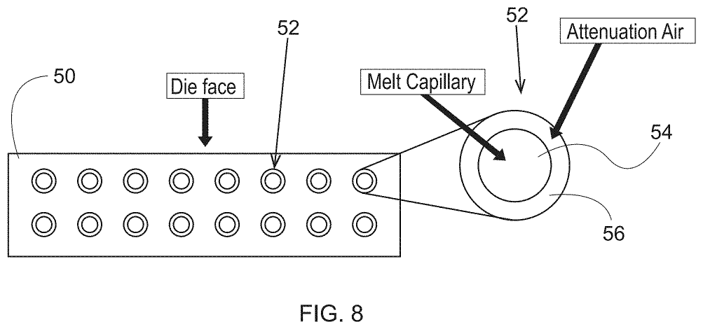

In one example, as shown in FIGS. 7 and 8 a method 46 for making a fibrous element 32 according to the present invention comprises the steps of:

a. providing a filament-forming composition 48 comprising one or polymeric structurants, and optionally one or more other ingredients including high melting point fatty materials and/or one or more surfactants; and

b. spinning the filament-forming composition 48, such as via a spinning die 50, into one or more fibrous elements 32, such as filaments, comprising the one or more polymeric structurants and optionally, the one or more other ingredients. The one or more other ingredients may be releasable from the fibrous element when exposed to conditions of intended use. The total level of the one or more polymeric structurants present in the fibrous element 32, may be less than 80% and/or less than 70% and/or less than 65% and/or 50% or less by weight on a dry fibrous element basis and/or dry fibrous structure basis and the total level of the one or more active agents, when present in the fibrous element may be greater than 20% and/or greater than 35% and/or 50% or greater 65% or greater and/or 80% or greater by weight on a dry fibrous element basis and/or dry fibrous structure basis.

As shown in FIG. 8, the spinning die 50 may comprise a plurality of fibrous element-forming holes 52 that include a melt capillary 54 encircled by a concentric attenuation fluid hole 56 through which a fluid, such as air, passes to facilitate attenuation of the filament-forming composition 48 into a fibrous element 32 as it exits the fibrous element-forming hole 52.

In one example, during the spinning step, any volatile solvent, such as water, present in the filament-forming composition 48 is removed, such as by drying, as the fibrous element 32 is formed. In one example, greater than 30% and/or greater than 40% and/or greater than 50% and/or greater than 60% of the weight of the filament-forming composition's volatile solvent, such as water, is removed during the spinning step, such as by drying the fibrous element being produced.

The filament-forming composition may comprise any suitable total level of polymeric structurant and any suitable level of active agents so long as the fibrous element produced from the filament-forming composition comprises a total level of polymeric structurant in the fibrous element of from about 5% to 50% or less by weight on a dry fibrous element basis and/or dry fibrous structure basis and a total level of active agents in the fibrous element of from 50% to about 95% by weight on a dry fibrous element basis and/or dry fibrous structure basis.

In one example, the filament-forming composition may comprise any suitable total level of polymeric structurant and any suitable level of active agents so long as the fibrous element produced from the filament-forming composition comprises a total level of polymeric structurant in the fibrous element and/or particle of from about 5% to 50% or less by weight on a dry fibrous element basis and/or dry fibrous structure basis and a total level of active agents in the fibrous element and/or particle of from 50% to about 95% by weight on a dry fibrous element basis and/or dry fibrous structure basis, wherein the weight ratio of polymeric structurant to total level of surfactant and/or high melting point fatty material is 1 or less.

In one example, the filament-forming composition comprises from about 1% and/or from about 5% and/or from about 10% to about 50% and/or to about 40% and/or to about 30% and/or to about 20% by weight of the filament-forming composition of polymeric structurant; from about 1% and/or from about 5% and/or from about 10% to about 50% and/or to about 40% and/or to about 30% and/or to about 20% by weight of the filament-forming composition of active agents; and from about 20% and/or from about 25% and/or from about 30% and/or from about 40% and/or to about 80% and/or to about 70% and/or to about 60% and/or to about 50% by weight of the filament-forming composition of a volatile solvent, such as water. The filament-forming composition may comprise minor amounts of other active agents, such as less than 10% and/or less than 5% and/or less than 3% and/or less than 1% by weight of the filament-forming composition of plasticizers, pH adjusting agents, and other active agents.

The filament-forming composition is spun into one or more fibrous elements and/or particles by any suitable spinning process, such as meltblowing, spunbonding, electro-spinning, and/or rotary spinning. In one example, the filament-forming composition is spun into a plurality of fibrous elements and/or particles by meltblowing. For example, the filament-forming composition may be pumped from a tank to a meltblown spinnerette. Upon exiting one or more of the filament-forming holes in the spinnerette, the filament-forming composition is attenuated with air to create one or more fibrous elements and/or particles. The fibrous elements and/or particles may then be dried to remove any remaining solvent used for spinning, such as the water.

The fibrous elements and/or particles of the present invention may be collected on a belt, such as a patterned belt to form a fibrous structure comprising the fibrous elements and/or particles.

### Test Methods

Unless otherwise specified, all tests described herein including those described under the Definitions section and the following test methods are conducted on samples that have been conditioned in a conditioned room at a temperature of 23° C.±1.0° C. and a relative humidity of 50%±2% for a minimum of 2 hours prior to the test. The samples tested are “usable units.” “Usable units” as used herein means sheets, flats from roll stock, pre-converted flats, and/or single or multi-ply products. All tests are conducted under the same environmental conditions and in such conditioned room. Do not test samples that have defects such as wrinkles, tears, holes, and like. Samples conditioned as described herein are considered dry samples (such as “dry filaments”) for testing purposes. All instruments are calibrated according to manufacturer's specifications.

### Basis Weight Test Method

Basis weight of a fibrous structure is measured on stacks of twelve usable units using a top loading analytical balance with a resolution of ±0.001 g. The balance is protected from air drafts and other disturbances using a draft shield. A precision cutting die, measuring 3.500 in±0.0035 in by 3.500 in±0.0035 in is used to prepare all samples.

With a precision cutting die, cut the samples into squares. Combine the cut squares to form a stack twelve samples thick. Measure the mass of the sample stack and record the result to the nearest 0.001 g.

The Basis Weight is calculated in lbs/3000 ft² or g/m² as follows:

Basis Weight=(Mass of stack)/[(Area of 1 square in stack)×(No. of squares in stack)]

For example,

Basis Weight (lbs/3000 ft²)=[[Mass of stack (g)/453.6 (g/lbs)]/[12.25 (in²)/144 (in²/ft²)×12]]×3000

or,

Basis Weight (g/m²)=Mass of stack (g)/[79.032 (cm²)/10,000 (cm²/m²)×12]

Report result to the nearest 0.1 lbs/3000 ft² or 0.1 g/m². Sample dimensions can be changed or varied using a similar precision cutter as mentioned above, so as at least 100 square inches of sample area in stack.

### Water Content Test Method

The water (moisture) content present in a fibrous element and/or particle and/or fibrous structure is measured using the following Water Content Test Method. A fibrous element and/or particle and/or fibrous structure or portion thereof (“sample”) in the form of a pre-cut sheet is placed in a conditioned room at a temperature of 23° C.±1.0° C. and a relative humidity of 50%±2% for at least 24 hours prior to testing. Each fibrous structure sample has an area of at least 4 square inches, but small enough in size to fit appropriately on the balance weighing plate. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into the forced air oven on top of foil for 24 hours at 70° C.±2° C. at a relative humidity of 4%±2% for drying. After the 24 hours of drying, the sample is removed and weighed within 15 seconds. This weight is designated as the “dry weight” of the sample.

The water (moisture) content of the sample is calculated as follows:

$$\% Water in sample = 100 \% \times \frac{( \begin{matrix}Equilibrium weight of sample - \\ Dry weight of sample\end{matrix} )}{Dry weight of sample}$$

The % Water (moisture) in sample for 3 replicates is averaged to give the reported % Water (moisture) in sample. Report results to the nearest 0.1%.

### Thickness Test Method

Thickness of a fibrous structure is measured by cutting 5 samples of a fibrous structure sample such that each cut sample is larger in size than a load foot loading surface of a VIR Electronic Thickness Tester Model II available from Thwing-Albert Instrument Company, Philadelphia, Pa. Typically, the load foot loading surface has a circular surface area of about 3.14 in². The sample is confined between a horizontal flat surface and the load foot loading surface. The load foot loading surface applies a confining pressure to the sample of 15.5 g/cm². The thickness of each sample is the resulting gap between the flat surface and the load foot loading surface. The thickness is calculated as the average thickness of the five samples. The result is reported in millimeters (mm).

### Diameter Test Method

The diameter of a discrete fibrous element or a fibrous element within a fibrous structure is determined by using a Scanning Electron Microscope (SEM) or an Optical Microscope and an image analysis software. A magnification of 200 to 10,000 times is chosen such that the fibrous elements are suitably enlarged for measurement. When using the SEM, the samples are sputtered with gold or a palladium compound to avoid electric charging and vibrations of the fibrous element in the electron beam. A manual procedure for determining the fibrous element diameters is used from the image (on monitor screen) taken with the SEM or the optical microscope. Using a mouse and a cursor tool, the edge of a randomly selected fibrous element is sought and then measured across its width (i.e., perpendicular to fibrous element direction at that point) to the other edge of the fibrous element. A scaled and calibrated image analysis tool provides the scaling to get actual reading in μm. For fibrous elements within a fibrous structure, several fibrous element are randomly selected across the sample of the fibrous structure using the SEM or the optical microscope. At least two portions of the fibrous structure are cut and tested in this manner. Altogether at least 100 such measurements are made and then all data are recorded for statistical analysis. The recorded data are used to calculate average (mean) of the fibrous element diameters, standard deviation of the fibrous element diameters, and median of the fibrous element diameters.

Another useful statistic is the calculation of the amount of the population of fibrous elements that is below a certain upper limit. To determine this statistic, the software is programmed to count how many results of the fibrous element diameters are below an upper limit and that count (divided by total number of data and multiplied by 100%) is reported in percent as percent below the upper limit, such as percent below 1 micrometer diameter or %-submicron, for example. We denote the measured diameter (in μm) of an individual circular fibrous element as di.

In the case that the fibrous elements have non-circular cross-sections, the measurement of the fibrous element diameter is determined as and set equal to the hydraulic diameter which is four times the cross-sectional area of the fibrous element divided by the perimeter of the cross-section of the fibrous element (outer perimeter in case of hollow fibrous elements). The number-average diameter, alternatively average diameter is calculated as:

$${d}_{num} = \frac{{\sum }_{i = 1}^{n} {d}_{i}}{n}$$

### Fibrous Element Composition Test Method

In order to prepare fibrous elements for fibrous element composition measurement, the fibrous elements must be conditioned by removing any coating compositions and/or materials present on the external surfaces of the fibrous elements that are removable. An example of a method for doing so is washing the fibrous elements 3 times with a suitable solvent that will remove the external coating while leaving the fibrous elements unaltered. The fibrous elements are then air dried at 23° C.±1.0° C. until the fibrous elements comprise less than 10% moisture. A chemical analysis of the conditioned fibrous elements is then completed to determine the compositional make-up of the fibrous elements with respect to the filament-forming materials and the active agents and the level of the filament-forming materials and active agents present in the fibrous elements.

The compositional make-up of the fibrous elements with respect to the filament-forming material and the active agents can also be determined by completing a cross-section analysis using TOF-SIMs or SEM. Still another method for determining compositional make-up of the fibrous elements uses a fluorescent dye as a marker. In addition, as always, a manufacturer of fibrous elements should know the compositions of their fibrous elements.

### Hand Dissolution Test Method

### Materials Needed:

Fibrous structures to be tested: 3-5 fibrous structures (finished product samples) are tested so that an average of the number of strokes for each if the individual fibrous structure samples is calculated and recorded as the Average Hand Dissolution value for the fibrous structure. For this method, the entire consumer saleable or consumer use fibrous structure is tested. If the entire consumer saleable or consumer use fibrous structure has a footprint greater than 50 cm², then first cut the fibrous structure to have a footprint of 50 cm².

Nitrile Gloves

10 cc syringe

Plastic Weigh boat (˜3 in×3 in)

100 mL Glass beaker

Water (City of Cincinnati Water or equivalent having the following properties: Total Hardness=155 mg/L as CaCO₂; Calcium content=33.2 mg/L; Magnesium content=17.5 mg/L; Phosphate content=0.0462 mg/L). Water used is water 7 grains per gallon (gpg) hardness and 40° C.+/−5° C.

### Protocol:

Add 80 mL of water to glass beaker.
Heat water in beaker until water is at a temperature of 40° C.+/−5° C.
Transfer 15 mL of the water from the beaker into the weigh boat via the syringe.
Within 10 seconds of transferring the water to the weigh boat, place fibrous structure sample in palm of gloved hand (hand in cupped position in non-dominant hand to hold fibrous structure sample).
Using dominant hand, add water quickly from the weigh boat to the fibrous structure sample and allow to immediately wet for a period of 5-10 seconds.
Rub with opposite dominant hand (also gloved) in 2 rapid circular strokes.
Visually examine the fibrous structure sample in hand after the 2 strokes. If fibrous structure sample is completely dissolved, record number of strokes=2 Dissolution Strokes. If not completely dissolved, rub remaining fibrous structure sample for 2 more circular strokes (4 total) and observe degree of dissolution. If the fibrous structure sample contains no solid pieces after the 2 additional strokes, record number of strokes=4 Dissolution Strokes. If after the 4 strokes total, the fibrous structure sample still contains solid pieces of un-dissolved fibrous structure sample, continue rubbing remaining fibrous structure sample in additional 2 circular strokes and check if there are any remaining solid pieces of fibrous structure sample after each additional 2 strokes until fibrous structure sample is completely dissolved or until reaching a total of 30 strokes, whichever comes first. Record the total number of strokes. Record 30 Dissolution Strokes even if solid fibrous structure sample pieces remain after the maximum of 30 strokes.
Repeat this process for each of the additional 4 fibrous structure samples.
Calculate the arithmetic mean of the recorded values of Dissolution Strokes for the 5 individual fibrous structure samples and record as the Average Hand Dissolution Value for the fibrous structure. The Average Hand Dissolution Value is reported to the nearest single Dissolution Stroke unit.

### EXAMPLES

The following are non-limiting examples of primary packages containing solid articles described herein. It will be appreciated that other modifications of the present invention within the skill of those in the art can be undertaken without departing from the spirit and scope of this invention.

All parts, percentages, and ratios herein are by weight unless otherwise specified. Some components may come from suppliers as dilute solutions. The amount stated reflects the weight percent of the added material, unless otherwise specified.

The Examples in Table 1, below, were made as follows. First, a fibrous element-forming composition was prepared by adding water to a container under sufficient stirring, then adding the polyvinyl alcohol polymer(s). The mixture was heated to about 75° C. for about 2-3 hours until a homogenous and smooth polymer solution was formed. Then, the surfactant and other active ingredients are added one by one to the smooth polymer solution with mixing until a homogeneous solution was obtained. Additional ingredients, except for the sodium bicarbonate were subsequently added. The resulting mixture was stirred until a uniform mixture was obtained. The mixture is then allowed to degas, and the resulting viscous smooth mixture is used to form the fibrous elements and structures according to the Method for Making Fibrous Elements and Structures described herein. Lastly, the sodium bicarbonate was added as agglomerated particles.

Example A was used for Trials 1-6, described below. In Trial 1, the fibrous article was an oval and in Trials 3-6 the fibrous article was a regular hexagon. In all trials, the fibrous article had a base surface area of 17.82 cm² (2.76 in²) and a height of 5 mm (0.20 in.).

TABLE 1
Examples of Fibrous Articles

| | Example A (% by mass |
|---|---|
| | of fibrous article) |
| USP Water | 3.13% |
| Guar hydroxypropyltrimonium chloride¹ | 0.81 |
| Polyquaternium-76² | 0.16% |
| Polyvinyl alcohol³ | 10.27 |
| Polyvinyl alcohol⁴ | 10.27 |
| Citric Acid | 8.48 |
| Lauryl hydroxysultaine⁵ | 12.16 |
| Sodium Laureth 1 Sulfate | 19.03 |
| Sodium Laureth-3 Sulfate | 2.59 |
| Sodium Undecyl Sulfate | 11.60 |
| Sodium Bicarbonate | 8.83 |
| Amodimethicone⁶ | 4.63-9.26 |
| Fragrance and other minors | Q.S. |

¹Jaguar C500 supplied by Solvay ®
²Mirapol ® AM-T supplied by Solvay ®
³Poval ™ 3-80³, PVA420H supplied by Kuraray ®
⁴Poval ™ 32-80 (420H), PVA403 supplied by Kuraray ®
⁵Mackham ® LHS supplied by Solvay ®
⁶Y-14945 Amino Fluid supplied by Momentive ®

In the Trials described below, dissolution (# of strokes) was determined by the Hand Dissolution Test Method, as described herein.

### Trial 1: Effect of Compression on Articles Containing Fibrous Structures

To test the effect of compression the fibrous structures, gauging plates were constructed with precision milled and lapped spacers of the following thicknesses: 0.045 in., 0.040 in., 0.035 in., 0.030 in., 0.025 in., 0.020 in. A fibrous article was placed between the gauging plates and compressed to a given thickness. A minimum of three fibrous articles were tested at each thickness. The fibrous articles were removed from the plates with a spatula and immediately tested for hand dissolution following the Hand Dissolution Test Method, described hereafter. The results from this test are in Table 2, below.

TABLE 2

| | Dissolution | Standard |
|---|---|---|
| Article | (# of strokes) | Deviation |
| 5 mm (no compression) | 8.7 | 1.2 |
| 4.5 mm (compressed 0.5 mm/ | 9.3 | 1.2 |
| compressed 10%) | | |
| 4.0 mm (compressed 1.0 mm/ | 12.7 | 2.3 |
| compressed 20%) | | |
| 3.5 mm (compressed 1.5 mm/ | 13.3 | 7.6 |
| compressed 30%) | | |
| 3.0 mm (compressed 2 mm/ | 19.3 | 4.2 |
| compressed 40%) | | |
| 2.5 mm (compressed 2.5 mm/ | 30 or more | n/a |
| 50% compressed) | | |
| 2.0 mm (compressed 3 mm/ | 30 or more | n/a |
| 60% compressed) | | |

It was found that the 5 mm (0.20 in.) articles were fluffy, hydrated easily, and produced lots of lather and cream during the Hand Dissolution Test. The 4.5 mm (0.18 in.) articles had consumer acceptable dissolution rate and while still consumer acceptable, it was observed that the lather fell off slightly faster than the 5 mm (0.20 in.), uncompressed article. The 4.0 mm (0.016 in.) articles also had consumer acceptable dissolution rates, and while still being consumer acceptable, in some articles there were small chunks that were slightly more difficult to hydrate. For the 3.5 mm (0.14 in.) and 3.0 mm (0.12 in.) articles the average dissolution was slower and less consistent and therefore not consumer acceptable. In some of these articles, there were regions of the article that took significant effort to dissolve and, in some instances, there were regions did not dissolve. The 2.5 mm (0.098 in.) and 2.0 mm (0.079 in.) articles had poor hand dissolution and after 30 strokes in the Hand Dissolution Test, they were still not fully dissolved. In some instances, the articles they did not hydrate at all and/or delaminated.

It was found that the number of hand strokes required for the fibrous structure to dissolve was directly proportional to the amount of compression of the article. For fibrous structures, as described herein, it was determined that if the article was compressed from 5 mm (0.20 in.) to 3.5 mm (0.14 in.) (i.e. a 30% reduction), it would take too long to disintegrate into a homogeneous solution and would not be consumer preferred. Therefore, the primary package needs to prevent the solid articles from being compressed by 1.5 mm (0.059 in.)/30% reduction in height.

### Trial 2: Comparison of Articles Stored in Weigh Boats Versus Solid Articles Sealed in Sachets

Many single use personal care products, including shampoo and conditioner, are stored in sachets. The sachets can be inexpensive, while providing an excellent moisture barrier. However, in addition to not being easily recycled, it was found that articles that were packaged and stored in sachets were compressed around the edges and when hydrated these articles would likely have clumps that are difficult to dissolve.

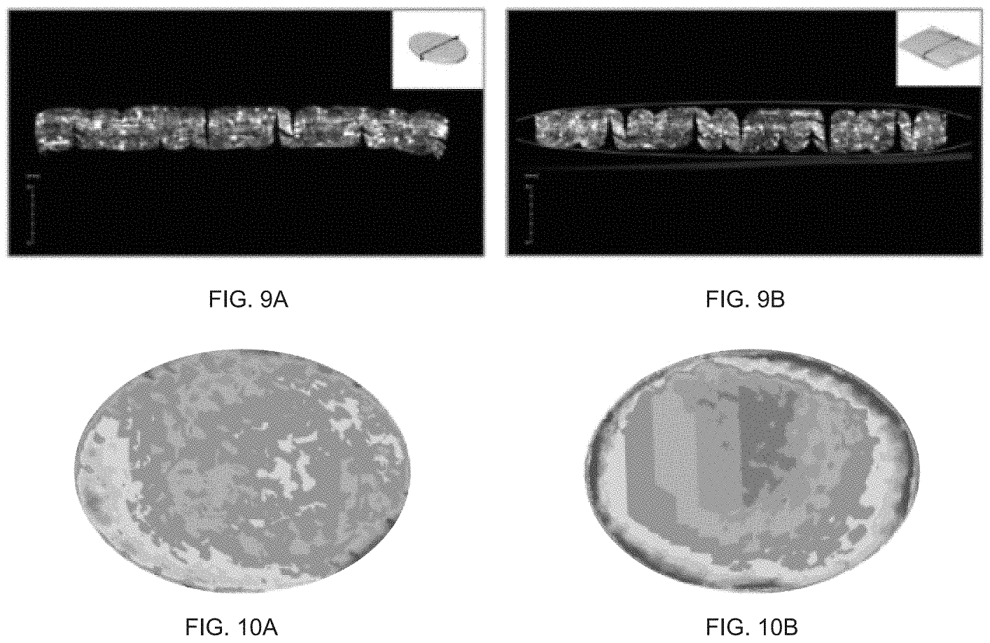

FIGS. 9A and 9B are computerized tomography (CT) scans of the fibrous articles of Example A, described in Table 1. FIG. 9A is an unpackaged article that was equilibrated in a weigh boat at ambient conditions (˜71-73° F. and ˜50% relative humidity) and FIG. 9B is a fibrous article was packaged in a foil sachet (material available from Mondi Jackson LLC). The unpackaged sachets were scanned outside the weigh boat, while the articles in the sachet were scanned inside the sachet. The CT scans show that the example in FIG. 9A is rectangular and square at the ends, while the article in FIG. 9B tapers at the ends.

FIGS. 10A-10B are 3D CT scans of the fibrous articles of Example A, described in Table 1. These CT scans use color to display degrees of compression with red color indicating compression of the article and the blue or bluer regions indicate less or no compression of the article. FIG. 10A is 3D CT scan of an unpackaged article that was stored in a weigh boat and FIG. 10B is a 3D CT scan of a fibrous article was packaged in a sachet. The red areas around the perimeter of FIG. 10B, show that the edges of the fibrous article are compressed.

Overall, Trial 2 shows that even if sachets could withstand the shipping, handling, and storage conditions that the solid article is likely to encounter, a sachet is not acceptable at the outset because the sealing process subjects the outside edges to too much pressure to allow for a smooth dissolution.

### Trial 3: Effect of Humidity on Articles Containing Fibrous Structures

To test the effect of humidity on fibrous articles, three fibrous articles were stacked together and placed in an open thermoform or weight boat to serve as the control. The samples were placed at various relative humidity and temperature conditions for four days, as described in Table 3, below. The fibrous articles were removed from the thermoform or weigh boat with a spatula and immediately tested for hand dissolution following the Hand Dissolution Test Method, described herein. The results are in Table 3, below.

TABLE 3

| | Temperature/ | | | |
|---|---|---|---|---|
| | Relative | | Dissolution | std |
| Ex. | Humidity | Packaging | (# of strokes) | dev |
| 1 | 73° F./40% | control in weigh boat | 8 | 1 |
| 2 | 73° F./40% | in thermoform with no lid | 8 | 1 |
| 3 | 104° F./20% | in thermoform with no lid | 10 | 2 |
| 4 | 80° F./80% | in thermoform with no lid | 29 | 2 |
| 5 | 77° F./60% | in thermoform with no lid | 10 | 2 |

Examples 2, 3, and 5 are stored between 20% and 60% relative humidity and have consumer acceptable dissolution, as determined by the Hand Dissolution. Example 4 was stored with a relative humidity of 80% and the hand dissolution was not acceptable. The inventors believe that this is due to gel blocking, which occurs when the outside of the fibrous article gets wet and forms a gel, ultimately preventing water from reaching the interior fibers and preventing dissolution.

Since the primary package with the fibrous article will likely be stored in the shower, which was determined to have an average relative humidity of 68% at 68° F., it was determined that inside the thermoform compartment had a relative humidity between about 20% to about 60%, the consumer will still experience acceptable hand dissolution of the fibrous article.

### Trial 4: Thermoform MVTR

Different materials and thicknesses of thermoform were tested at different temperatures and relative humidity to determine what thermoforms could keep the relative humidity inside the thermoform compartment between 20-60%. An 11/16 in. circular hole was cut into the center of each fibrous article and the fibrous article was placed in the thermoform compartment. A data logger (iButton® temperature/humidity logger (DS1923)), available from Maxim Integrated™, San Jose, Calif., USA) was placed into the hole in the article before the lidding film was applied and sealed. The data logger measured the relative humidity over the time period. The following thermoform materials were tested:
0.020 in. polyethylene terephthalate glycol (PET-G)
0.030 in. PET-G
0.040 in. PET-G,
0.060 in. PET-G
0.006 in. Aclar®/0.001 PET laminate (poly-chloro-trifluoroethylene/polyethylene terephthalate glycol, available from Klockner Pentaplast, Gordonsville, Va., USA)

Each thermoform contained an article, as described in Table 1. The lidding film is sealed to the flange of the thermoform at conditions that form a heat seal. The lidding film was a laminate of aluminum foil (0.001 in.) and PET (0.0005 in.) (commercially available from Amcor®).

Each thermoform was tested three times at the following temperatures/relative humidity:
77° F./60% RH
86° F./65% RH
Consumer's Shower (on average about 68° F./68% RH)

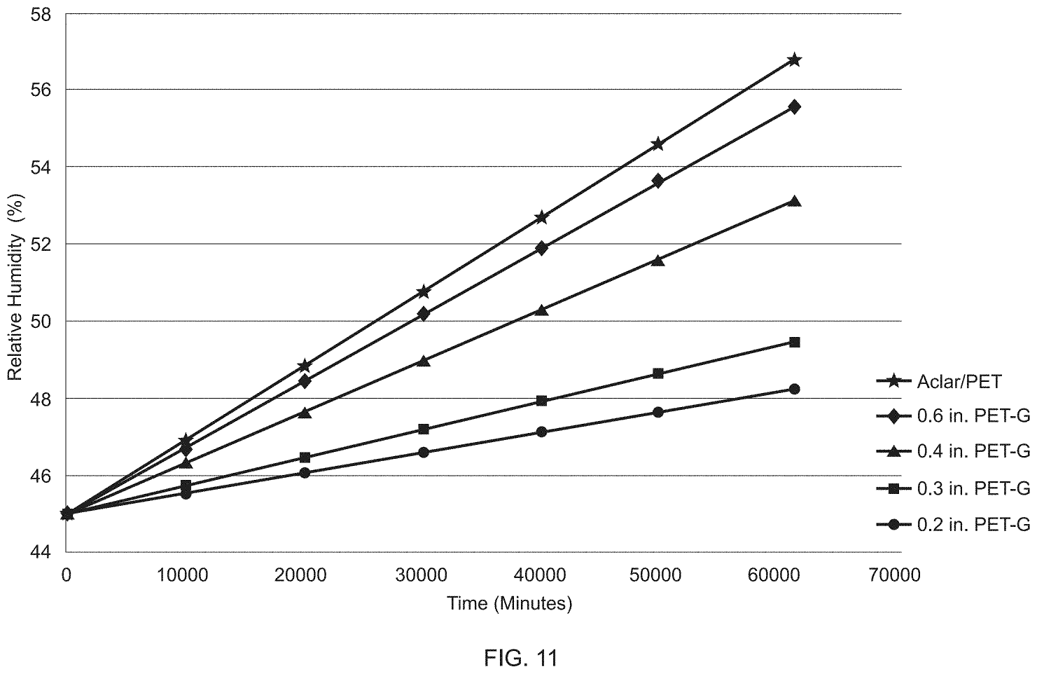

FIG. 11 is a chart showing the % relative humidity vs. time for the five thermoforms that were tested. All the thermoforms tested may be acceptable to consumers because the relative humidity inside the thermoform compartment does not rise above 60%.

Trial 5: 0.040 in. PET Thermoform MVTR

0.040 in. PET thermoforms each with a lidding film containing a laminate of aluminum foil (0.001 in.) and PET (0.0005 in.) (commercially available from Amcor®). The lidding film is sealed to the flange of the thermoform at 347° F. for 3 seconds at 30 psi to form a 3 mm (0.12 in.) heat seal. An 11/16 in. circular hole was cut into the center of each fibrous article and the fibrous article was placed in the thermoform compartment. A data logger (iButton® temperature/humidity logger (DS1923), available from Maxim Integrated™, San Jose, Calif., USA) was placed into the hole in the article before the lidding film was applied and sealed. The data logger measured the relative humidity over the time period. The thermoforms were aged for one week or two weeks at 40° C. (104° F.)/75% RH, which is an accelerated stability condition meant to represent eight weeks of in-shower conditions.

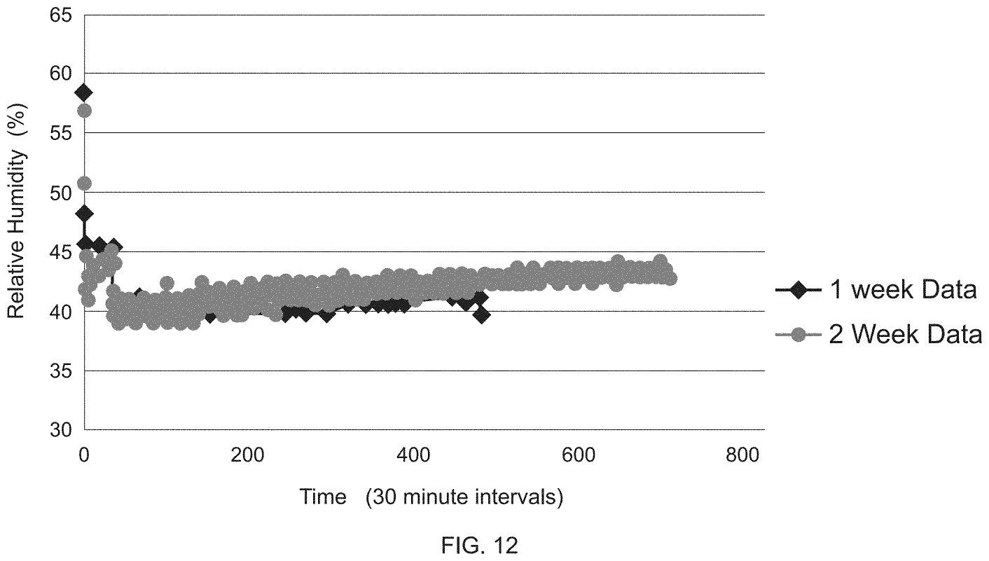

FIG. 12 is a chart showing the relative humidity inside the thermoform compartment vs. time. FIG. 12 shows that the 0.040 in. PET thermoform with laminate lidding film provided a sufficient moisture barrier because the relative humidity is significantly less than 60% for both the one-week test and the two-week test.

### Trial 6:

Fibrous articles with a base with an area of 2.68 in² were stored for 24 hours in each of the following conditions: 80° F./80% RH, 77° F./60% RH, 73° F./40% RH, and the 40° C. (104° F.)/20% RH. After 24 hours, the articles were removed and sealed in metalized mylar bags during transport to KV. The articles were compressed using a universal testing machine (KV's Instron®) at a speed of 50 mm/min and held for 15 seconds once the set height was reached.

As discussed above, it was determined that if the fibrous articles are compressed to more than 1.5 mm (30%), the dissolution is unacceptable. Table 4, below, shows the maximum force that can be exerted on the fibrous article to maintain this consumer acceptable dissolution.

TABLE 4

| Temperature/ | | |
|---|---|---|
| Relative Humidity | Force (lbf) | Compressive Force (psi) |
| 80° F./80% | 0 | 0 |
| 77° F./60% | 4.5 | 1.7 |
| 73° F./40% | 15.7 | 5.9 |
| 104° F./20% | 40.5 | 15.1 |

Table 4 shows that the amount of compressive force that is required to achieve unacceptable consumer dissolution is dependent upon the relative humidity. Thus, if the relative humidity inside the thermoform compartment is 80%, the filaments of the fibrous article collapse under their own weight, resulting in acceptable hand dissolution without any compressive forces. As the relative humidity decreases, the amount of force required to achieve unacceptable dissolution increases. Therefore, the fibrous articles need to be protected from compression and relative humidity.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care product comprising:
a. primary package comprising:
i. a thermoform portion comprising a peripheral flange and a thermoform compartment;
wherein the thermoform portion is comprised of a thermoform material selected from the group consisting of polypropylene, polyethylene terephthalate, polyethylene terephthalate glycol, poly-chloro-trifluoroethylene/polyethylene terephthalate glycol, and combinations thereof;
ii. a lidding film peelably sealed to the peripheral flange;
wherein the primary package comprises a moisture vapor transmission rate of less than 0.25 g/m²/day;
b. a solid fibrous article comprising filaments comprising:
i. from about 1 wt % to about 50 wt % of a polymeric structurant having a weight average molecular weight of from about 10,000 to about 6,000,000 g/mol;
ii. a surfactant selected from the group consisting of anionic surfactants, cationic surfactants, zwitterionic surfactants, and combinations thereof;
wherein the fibrous article fits within the thermoform compartment;
wherein the fibrous article compresses less than 30% under a compressive force of at least 15 psi;
c. a headspace disposed between the lidding layer and a top surface of the solid fibrous article wherein the headspace comprises a height of from about 5% to about 30%, of the height of the thermoform.

2. The personal care product of claim 1 wherein the lidding film comprises a multilayer laminate comprising at least one of the following: aluminum foil, polyethylene terephthalate.

3. The personal care product of claim 1 wherein the thermoform material is selected from the group consisting of polypropylene, polyethylene terephthalate, and combinations thereof.

4. The personal care product of claim 1 wherein the filaments comprise from about 1 wt. % to about 60 wt. % cationic surfactant.

5. The personal care product of claim 1 wherein the filaments comprise from about 10 wt. % to about 90 wt. % anionic surfactant and wherein the structurant comprises polyvinyl alcohol.

6. The personal care product of claim 1 wherein the moisture vapor transmission rate is less than 0.20 g/m²/day.

7. The personal care product of claim 6 wherein the moisture vapor transmission rate is less than 0.15 g/m²/day.

8. The personal care product of claim 1 wherein the relative humidity inside the thermoform compart of the primary package is less than 60% after storage in the primary package for one week at 104° F. and 75% relative humidity.

9. The personal care product of claim 1 wherein the dissolvable fibrous article comprises a hand dissolution of less than or equal to 20 strokes, after storage in the primary package for one week at 104° F. and 75% relative humidity, according to the Hand Dissolution Test Method.

10. The personal care product of claim 2 wherein the dissolvable fibrous article comprises a hand dissolution of less than or equal to 15 strokes, after storage in the primary package for one week at 104° F. and 75% relative humidity, according to the Hand Dissolution Test Method.

11. The personal care product of claim 1 wherein the dissolvable fibrous article comprises a hand dissolution of less than or equal to 20 strokes, after storage in the primary package for six weeks at 104° F. and 75% relative humidity, according to the Hand Dissolution Test Method.

12. The personal care product of claim 11 wherein the dissolvable fibrous article comprises a hand dissolution of less than or equal to 15 strokes, after storage in the primary package for one week at 104° F. and 75% relative humidity, according to the Hand Dissolution Test Method.

13. The personal care product of claim 1 wherein the thermoform portion is transparent.

14. A personal care product comprising:
a. primary package comprising:
i. a thermoform portion comprising a peripheral flange and a thermoform compartment;
wherein the thermoform portion is comprised of a thermoform material selected from the group consisting of polypropylene, polyethylene terephthalate, polyethylene terephthalate glycol, poly-chloro-trifluoroethylene/polyethylene terephthalate glycol, and combinations thereof;
ii. a lidding film peelably sealed to the peripheral flange with a heat seal;
wherein the lidding film comprises a multilayer laminate wherein at least one layer is selected from the group consisting of aluminum, polyethylene terephthalate, and combinations thereof;
wherein the primary package comprises a moisture vapor transmission rate of less than 0.20 g/m²/day;
b. a solid article comprising a surfactant;
wherein the fibrous article fits within the thermoform compartment;
c. a headspace disposed between the lidding layer and a top surface of the solid article wherein the headspace comprises a height of from about 0.05 mm to about 1.5 mm;
wherein the primary package protects the solid article from compressing more than 30% under a compressive force of 50 N.

15. The personal care product of claim 14 wherein the primary package protects the solid article from compressing more than 30% under a compressive force of 100 N.

16. The personal care product of claim 14 wherein the lidding film defines a pull tab wherein the pull tab is not sealed to the thermoform portion.

17. The personal care product of claim 16 wherein the thermoform portion further comprises a grip portion adjacent to the heat seal wherein the grip portion is stepped out of plane of the peripheral flange by a transitional plane comprising a slope of less than 90 degrees with respect to the peripheral flange.

18. The personal care product of claim 14 wherein the thermoform portion comprises a sidewall comprising a thickness from about 0.020 in. to about 0.060 in.

19. The personal care product of claim 14 wherein the volume of the thermoformed compartment is from about 10% to about 25% larger than the volume of the solid article.

20. The personal care product of claim 14 wherein the moisture vapor transmission rate is less than 0.15 g/m2/day.
